(19) United States
(12) Patent Application Publication
(10) Pub. No.: US 2004/0077689 A1
(43) Pub. Date: Apr. 22, 2004

(54) Abc expression promoters

(76) Inventors: Yasuo Sugiyama, Kawanishi-shi (JP); Hiromatsu Fuse, Tsukuba-shi (JP); Masao Hirakata, Kobe-shi (JP); Ryuichi Tozawa, Toyonaka-shi (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/468,433

(22) PCT Filed: Apr. 24, 2002
(86) PCT No.: PCT/JP02/04072

(30) Foreign Application Priority Data
Apr. 25, 2001 (JP) 2001-128222

(51) Int. Cl. A61K 31/4439; A61K 31/44; A61K 31/426; A61K 31/195; A61K 31/40

(52) U.S. Cl. 514/342; 514/352; 514/354; 514/423; 514/426; 514/567; 514/369

(57) ABSTRACT

The ABCA1 mRNA expression-promoting agent, LXRα mRNA expression-promoting agent, ABCG1 mRNA expression-promoting agent, cholesterol efflux-promoting agent, cholesteryl ester accumulation-inhibiting agent, ACAT-1 mRNA expression-inhibiting agent and CEH mRNA expression-promoting agent of the present invention are excellent in the ability to control cholesterol distribution in the body and have low toxicity.

### TECHNICAL FIELD

The present invention relates to an ABCA1 mRNA expression promoting agent, an LXRα mRNA expression-promoting agent, an ABCG1 mRNA expression-promoting agent, a cholesterol efflux-promoting agent, a cholesteryl ester accumulation-inhibiting agent, an ACAT (acyl-coenzyme A: cholesterol acyl transferase)-1 mRNA expression-inhibiting agent and a CEH (cholesteryl ester hydrolase) mRNA expression-promoting agent that are useful for control of cholesterol distribution in the body.

### BACKGROUND ART

ABCA1 is a member of the. ATP-binding cassette-transporter family and is involved in control of apoA1-mediated cholesterol efflux from macrophages. ABCG1 is also involved in control of apoA1-mediated cholesterol efflux.

LXR-α is a nuclear receptor that is activated by oxysterol and mediates the transcription induction of ABCA1 and ABCG1 gene promoters.

Nature Medicine, Vol 7, No. 1, pp. 53-58 discloses that 1) PPAR-α and PPAR-γ activators induce expression of a gene encoding ABCA1, 2) PPAR-α and PPAR-γ activators increase cholesterol efflux from normal macrophages induced by apoA1, and 3) PPAR-α and PPAR-γ activators induce LXR-α gene expression in human macrophages, and exemplifies rosiglitazone and troglitazone as PPAR-γ activators.

There is need for development of an ABCA1 mRNA expression-promoting agent, an LXRα mRNA expression-promoting agent, an ABCG1 mRNA expression-promoting agent, a cholesterol efflux-promoting agent, a cholesteryl ester accumulation-inhibiting agent, an ACAT-1 mRNA expression-inhibiting agent and a CEH mRNA expression-promoting agent which have excellent ability to control cholesterol distribution in the body and have low toxicity.

### DISCLOSURE OF THE INVENTION

The present invention relates to:

1) an ABCA1 mRNA expression-promoting agent comprising pioglitazone or a salt thereof;

2) an ABCA1 mRNA expression-promoting agent comprising PPARδ modulator;

3) the agent according to the above-mentioned 2), wherein the PPARδ modulator is a compound represented by the formula:

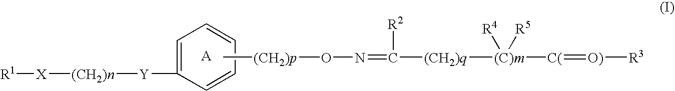

wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³ is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring [hereinafter sometimes abbreviated as compound (I)] or a salt thereof;

4) the agent according to the above-mentioned 3), wherein the PPARδ modulator is E-4-[4-(5-methyl-2-phenyl-4-oxazolylmethoxy)benzyloxyimino]-4-phenylbutyric acid [hereinafter sometimes abbreviated as compound (7)] or a salt thereof;

5) the agent according to the above-mentioned 2), wherein the PPARδ modulator is a compound represented by the formula:

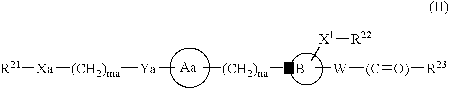

wherein R²¹ is an optionally substituted hydrocarbon group or an optionally substituted heterocycle group;

Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;

ma is an integer of 0 to 3;

Ya is an oxygen atom, a sulfur atom, —SO—, —SO₂—, —NR²⁷—, —CONR²⁷— or —NR²⁷CO—, wherein R²⁷ is a hydrogen atom or an optionally substituted hydrocarbon group;

ring Aa is an aromatic ring optionally having further 1 to 3 substituent(s);

na is an integer of 1 to 8;

ring B is a nitrogen-containing 5-membered heterocycle optionally further substituted with alkyl group(s);

X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;

R²² is a hydrogen atom, an optionally substituted hydrocarbon group or an optionally substituted heterocycle group;

W is a bond or a bivalent hydrocarbon residue having 1 to 20 carbon atom(s);

R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring [hereinafter sometimes abbreviated as compound (II)] or a salt thereof;

6) the agent according to the above-mentioned 5), wherein the PPARδ modulator is

3-[3-ethoxy-1-[4-(2-phenyl-4-thiazolylmethoxy)benzyl]-1H-pyrazol-4-yl]propionic acid;

3-[3-ethoxy-1-[4-(2-phenyl-4-oxazolylmethoxy)benzyl]-1H-pyrazol-4-yl]propionic acid;

3-[3-ethoxy-1-[4-[3-methyl-1-(2-pyridyl)-1H-pyrazol-4yl]methoxy]benzyl]-1H-pyrazol-4]propionic acid;

3-[1-[4-(5-methyl-2-phenyl-4-oxazolylmethoxy)benzyl]-3-(2-thienyl)-1H-pyrazol-4-yl]propionic acid;

3-[1-[4-(2-phenyl-4-thiazolylmethoxy)benzyl]-3-(2-thienyl)-1H-pyrazol-4-yl]propionic acid; or

3-[1-[4-(2-phenyl-4-oxazolylmethoxy)benzyl]-3-(2-thienyl)-1H-pyrazol-4-yl]propionic acid [hereinafter these compounds are sometimes abbreviated as compounds (11 to 16)];

7) an LXRα mRNA expression-promoting agent comprising pioglitazone or a salt thereof;

8) an LXRα mRNA expression-promoting agent comprising PPARδ modulator;

9) the agent according to the above-mentioned 8), wherein the PPARδ modulator is the compound (I) or a salt thereof;

10) the agent according to the above-mentioned 9), wherein the PPARδ modulator is the compound (7) or a salt thereof;

11) the agent according to the above-mentioned 8), wherein the PPARδ modulator is the compound (II) or a salt thereof;

12) the agent according to the above-mentioned 11), wherein the PPARδ modulator is any of the compounds (11 to 16);

13) an ABCG1 mRNA expression-promoting agent comprising pioglitazone or a salt thereof;

14) an ABCG1 mRNA expression-promoting agent comprising PPARδ modulator;

15) the agent according to the above-mentioned 14), wherein the PPARδ modulator is the compound (I) or a salt thereof;

16) the agent according to the above-mentioned 15), wherein the PPARδ modulator is the compound (7) or a salt thereof;

17) the agent according to the above-mentioned 14), wherein the PPARδ modulator is the compound (II) or a salt thereof;

18) the agent according to the above-mentioned 17), wherein the PPARδ modulator is any of the compounds (11 to 16);

19) an ABCG1 mRNA expression-promoting agent comprising a PPARα modulator;

20) a cholesterol efflux-promoting agent comprising pioglitazone or a salt thereof;

21) a cholesterol efflux-promoting agent comprising a PPARδ modulator;

22) the agent according to the above-mentioned 21), wherein the PPARδ modulator is the compound (I) or a salt thereof;

23) the agent according to the above-mentioned 22), wherein the PPARδ modulator is the compound (7) or a salt thereof;

24) the agent according to the above-mentioned 21), wherein the PPARδ modulator is the compound (II) or a salt thereof;

25) the agent according to the above-mentioned 24), wherein the PPARδ modulator is any of the compounds (11 to 16);

26) a cholesteryl ester accumulation-inhibiting agent comprising pioglitazone or a salt thereof;

27) a cholesteryl ester accumulation-inhibiting agent comprising a PPARδ modulator;

28) the agent according to the above-mentioned 27), wherein the PPARδ modulator is the compound (I) or a salt thereof;

29) the agent according to the above-mentioned 28), wherein the PPARδ modulator is the compound (7) or a salt thereof;

30) the agent according to the above-mentioned 27), wherein the PPARδ modulator is the compound (II) or a salt thereof;

31) the agent according to the above-mentioned 30), wherein the PPARδ modulator is any of the compounds (11 to 16);

32) an ACAT-1 mRNA expression-inhibiting agent comprising PPARγ modulator;

33) the agent according to the above-mentioned 32), wherein the PPARγ modulator is pioglitazone or a salt thereof;

34) the agent according to the above-mentioned 32), wherein the PPARγ modulator is the compound (I) or a salt thereof;

35) the agent according to the above-mentioned 34), wherein the PPARγ modulator is the compound (7) or a salt thereof;

36) the agent according to the above-mentioned 32), wherein the PPARγ modulator is the compound (II) or a salt thereof;

37) the agent according to the above-mentioned 36), wherein the PPARγ modulator is any of the compounds (11 to 16);

38) a CEH mRNA expression-promoting agent comprising a PPARγ modulator;

39) the agent according to the above-mentioned 38), wherein the PPARγ modulator is pioglitazone or a salt thereof;

40) the agent according to the above-mentioned 38), wherein the PPARγ modulator is the compound (I) or a salt thereof;

41) the agent according to the above-mentioned 40), wherein the PPARγ modulator is the compound (7) or a salt thereof;

42) the agent according to the above-mentioned 38), wherein the PPARγ modulator is the compound (II) or a salt thereof;

43) the agent according to the above-mentioned 42), wherein the PPARγ modulator is any of the compounds (11 to 16); and the like.

Salts of pioglitazone used in the present invention include pharmacologically acceptable salts such as salts with inorganic bases, salts with organic bases, salts with inorganic acids, salts with organic acids, salts with basic or acidic amino acids and the like.

Preferable examples of salts with inorganic bases include salts with alkaline metals such as sodium and potassium, alkali earth metals such as calcium and magnesium, aluminum, ammonium and the like.

Preferable examples of salts with organic bases include salts with trimethylamine, triethylamine, pyridine, picoline, ethanolamine, diethanolamine, triethanolamine, dicyclohexylamine, N,N-dibenzylethylenediamine and the like.

Preferable examples of salts with inorganic acids include salts with hydrochloric acid, hydrobromic acid, nitric acid, sulfuric acid, phosphoric acid and the like.

Preferable examples of salts-with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of salts with basic amino acids include salts with arginine, lysine, ornithine and the like. Preferable examples of salts with acidic amino acids include salts with aspartic acid, glutamic acid and the like.

Among the above-mentioned salts, the salt with inorganic acid is preferable and hydrochloride is especially preferable.

A “PPARδ modulator” used for the present invention may be any substance capable of activating or suppressing the function of peroxisome proliferator-activated receptor delta (PPARδ), and specific examples thereof include PPARδ agonists (including partial agonists), PPARδ antagonists (including partial antagonists) and the like. Furthermore, the “PPARδ modulator” is preferably a substance that shows obvious effect in vitro at a concentration of 10 μM or less.

Preferable examples of the “PPARδ modulator” include the above-mentioned “compound (I) or a salt thereof”, the above-mentioned “compound (II) or a salt thereof”, carbaprostacyclin(cPGI), L-165041 (Journal Biological Chemistry, 274, 6718-6728 (1999 Merck)), GW501516 (Proc Natl Acad Sci U.S.A. 2001 98(9): 5306-11) and the like. Among them, the “compound (I) or a salt thereof” and “compound (II) or a salt thereof” and the like are preferred.

In the present invention, the “PPARδ modulator” may be a substance identical to the below-mentioned “PPARγ modulator” and/or “PPARα modulator”.

Hereinafter a definition for each symbol in the formula (I) is explained in detail.

The hydrocarbon group of the “optionally substituted hydrocarbon group” represented by R¹ includes an aliphatic hydrocarbon group, an alicyclic hydrocarbon group, an alicyclic-aliphatic hydrocarbon group, an aromatic aliphatic hydrocarbon group and an aromatic hydrocarbon group. The number of carbon atoms contained in the hydrocarbon group is preferably 1 to 14.

The aliphatic hydrocarbon group is preferably an aliphatic hydrocarbon group having 1 to 8 carbon atom(s). The aliphatic hydrocarbon group includes a saturated aliphatic hydrocarbon group having 1 to 8 carbon atom(s) (e.g., an alkyl group and the like) such as methyl, ethyl, propyl, isopropyl, butyl, isobutyl, sec.-butyl, t.-butyl, pentyl, isopentyl, neopentyl, hexyl, isohexyl, heptyl, octyl and the like; and an unsaturated aliphatic hydrocarbon group having 2 to 8 carbon atoms (e.g., an alkenyl group having 2 to 8 carbon atoms, an alkadienyl group having 4 to 8 carbon atoms, an alkenylalkynyl group having 2 to 8 carbon atoms, an alkadiynyl group having 4 to 8 carbon atoms and the like) such as ethenyl, 1-propenyl, 2-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 3-hexenyl, 2,4-hexadienyl, 5-hexenyl, 1-heptenyl, 1-octenyl, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 2,4-hexadiynyl, 5-hexynyl, 1-heptynyl, 1-octynyl and the like.

The alicyclic hydrocarbon group is preferably an alicyclic hydrocarbon group having 3 to 7 carbon atoms. The alicyclic hydrocarbon group include a saturated alicyclic hydrocarbon group having 3 to 7 carbon atoms (e.g., a cycloalkyl group and the like) such as cyclopropyl, cyclobutyl, cyclopentyl, cyclohexyl, cycloheptyl and the like; and an unsaturated alicyclic hydrocarbon group having 5 to 7 carbon atoms (e.g., a cycloalkenyl group, a cycloalkadienyl group and the like) such as 1-cyclopentenyl, 2-cyclopentenyl, 3-cyclopentenyl, 1-cyclohexenyl, 2cyclohexenyl, 3-cyclohexenyl, 1-cycloheptenyl, 2-cycloheptenyl, 3-cycloheptenyl, 2,4-cycloheptadienyl and the like.

The alicyclic-aliphatic hydrocarbon group includes groups obtained by linking the above-mentioned alicyclic hydrocarbon group and aliphatic hydrocarbon group (e.g., a cycloalkyl-alkyl group, a cycloalkenyl-alkyl group and the like). Among them, an alicyclic-aliphatic hydrocarbon group having 4 to 9 carbon atoms is preferred. The alicyclic-aliphatic hydrocarbon group includes cyclopropylmethyl, cyclopropylethyl, cyclobutylmethyl, cyclopentylmethyl, 2-cyclopentenylmethyl, 3-cyclopentenylmethyl, cyclohexylmethyl, 2-cyclohexenylmethyl, 3-cyclohexenylmethyl, cyclohexylethyl, cyclohexylpropyl, cycloheptylmethyl, cycloheptylethyl and the like.

The aromatic aliphatic hydrocarbon group is preferably an aromatic aliphatic hydrocarbon group having 7 to 13 carbon atoms (e.g., an aralkyl group having 7 to 13 carbon atoms, an arylalkenyl group having 8 to 13 carbon atoms and the like). The aromatic aliphatic hydrocarbon group includes phenylalkyl having 7 to 9 carbon atoms such as benzyl, phenethyl, 1-phenylethyl, 1-phenylpropyl, 2-phenylpropyl, 3-phenylpropyl and the like; naphthylalkyl having 11 to 13 carbon atoms such as α-naphthylmethyl, α-naphthylethyl, β-naphthylmethyl, β-naphthylethyl and the like; phenylalkenyl having 8 to 10 carbon atoms such as styryl and the like; and naphthylalkenyl having 12 or 13 carbon atoms such as 2-(2-naphthylvinyl) and the like.

The aromatic hydrocarbon group is preferably an aromatic hydrocarbon group having 6 to 14 carbon atoms (e.g., aryl group and the like). The aromatic hydrocarbon group includes phenyl, naphthyl, anthryl, phenanthryl, acenaphthylenyl, biphenylyl and the like. Among them, phenyl, 1-naphthyl, 2-naphthyl and the like are preferred.

The heterocycle group of the “optionally substituted heterocycle group” represented by R¹ includes a 5- to 7-membered monocyclic heterocycle group containing 1 to 4 heteroatom(s) selected from an oxygen atom, a sulfur atom and a nitrogen atom as the ring-constituting atoms besides carbon atoms, or a fused heterocycle group. The fused heterocycle includes a fused ring of the 5- to 7-membered monocyclic heterocycle with a 6-membered ring containing 1 or 2 nitrogen atom(s), a benzene ring or a 5-membered ring containing one sulfur atom.

Specific examples of the heterocycle group include an aromatic heterocycle group such as 2-pyridyl, 3-pyridyl, 4-pyridyl, 2-pyrimidinyl, 4-pyrimidinyl, 5-pyrimidinyl, 6-pyrimidinyl, 3-pyridazinyl, 4-pyridazinyl, 2-pyrazinyl, 1-pyrrolyl, 2-pyrrolyl, 3-pyrrolyl, 1-imidazolyl, 2-imidazolyl, 4-imidazolyl, 5-imidazolyl, 1-pyrazolyl, 3-pyrazolyl, 4-pyrazolyl, isoxazolyl, isothiazolyl, 2-thiazolyl, 4-thiazolyl, 5-thiazolyl, 2-oxazolyl, 4-oxazolyl, 5-oxazolyl, 1,2,4-oxadiazol-5-yl, 1,3,4-oxadiazol-2-yl, 1,3,4-thiadiazol-2-yl, 1,2,4-triazol-1-yl, 1,2,4-triazol-3-yl, 1,2,3-triazol-1-yl, 1,2,3-triazol-2-yl, 1,2,3-triazol-4-yl, tetrazol-1-yl, tetrazol-5-yl, 2-quinolyl, 3-quinolyl, 4-quinolyl, 2-quinazolyl, 4-quinazolyl, 2-quinoxalyl, 2-benzoxazolyl, 2-benzothiazolyl, benzimidazol-1-yl, benzimidazol-2-yl, indol-1-yl, indol-3-yl, 1H-indazol-3-yl, 1H-pyrrolo[2,3-b]pyrazin-2-yl, 1H-pyrrolo[2,3-b]pyridin-6-yl, 1H-imidazo[4,5-b]pyridin-2-yl, 1H-imidazo[4,5c]pyridin-2-yl, 1H-imidazo[4,5-b]pyrazin-2-yl and the like; and a nonaromatic heterocycle group such as 1-pyrrolidinyl, piperidino, morpholino, thiomorpholino, 1-piperazinyl, hexamethyleneimin-1-yl, oxazolidin-3-yl, thiazolidin-3-yl, imidazolidin-3-yl, 2-oxoimidazolidin-1-yl, 2,4-dioxoimidazolidin-3-yl, 2,4-dioxooxazolidin-3-yl, 2,4-dioxothiazolidin-3-yl and the like.

The heterocycle group is preferably pyridyl, oxazolyl, thiazolyl, benzoxazolyl or benzothiazolyl.

Each of the hydrocarbon group and heterocycle group represented by R¹ may have 1 to 5, preferably 1 to 3 substituent(s) at the substitutable position(s). The substituents include an optionally substituted aliphatic hydrocarbon group, an optionally substituted alicyclic hydrocarbon group, an optionally substituted aromatic hydrocarbon group, an optionally substituted aromatic heterocycle group, an optionally substituted nonaromatic heterocycle group, a halogen atom, a nitro group, an optionally substituted amino group, an optionally substituted acyl group, an optionally substituted hydroxy group, an optionally substituted thiol group, an optionally esterified or amidated carboxyl group.

The substituents for the “optionally substituted aliphatic hydrocarbon group”, “optionally substituted alicyclic hydrocarbon group”, “optionally substituted aromatic hydrocarbon group”, “optionally substituted aromatic heterocycle group” and “optionally substituted nonaromatic heterocycle group” include a C₁₋₆ alkyl group, a C₁₋₆ alkoxy group, a halogen atom (e.g., fluorine, chlorine, bromine, iodine and the like), a nitro group, a C₁₋₆ haloalkyl group and a C₁₋₆ haloalkoxy group. The number of the substituents is, for example, 1 to 3.

The aliphatic hydrocarbon group includes a straight or branched chain aliphatic hydrocarbon group having 1 to 15 carbon atom(s) such as an alkyl group, an alkenyl group, an alkynyl group and the like.

Preferable examples of the alkyl group include an alkyl group having 1 to 10 carbon atom(s) such as methyl, ethyl, propyl, isopropyl, butyl, isobutyl, sec.-butyl, t.-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like.

Preferable examples of the alkenyl group include an alkenyl group having 2 to 10 carbon atoms such as ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like.

Preferable examples of the alkynyl group include an alkynyl group 2 to 10 carbon atoms such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like.

The alicyclic hydrocarbon group includes a saturated or unsaturated alicyclic hydrocarbon group having 3 to 12 carbon atoms such as a cycloalkyl group, a cycloalkenyl group, a cycloalkadienyl group and the like.

Preferable examples of the cycloalkyl group include a cycloalkyl group having 3 to 10 carbon atoms such as cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl and the like.

Preferable examples of the cycloalkenyl group include a cycloalkenyl group having 3 to 10 carbon atoms such as 2-cyclopenten-1-yl, 3-cyclopenten-1-yl, 2-cyclohexen-1-yl, 3-cyclohexen-1-yl and the like.

Preferable examples of the cycloalkadienyl group include a cycloalkadienyl group having 4 to 10 carbon atoms such as 2,4-cyclopentadien-1-yl, 2,4-cyclohexadien-1-yl, 2,5-cyclohexadien-1-yl and the like.

Preferable examples of the aromatic hydrocarbon group include an aromatic hydrocarbon group having 6 to 14 carbon atoms (e.g., aryl group and the like) such as phenyl, naphthyl, anthryl, phenanthryl, acenaphthylenyl, biphenylyl and the like. Among them, phenyl, 1-naphthyl, 2-naphthyl and the like are preferred.

Preferable examples of the aromatic heterocycle group include a 5- to 7-membered aromatic monocyclic heterocycle group containing 1 to 4 heteroatom(s) selected from an oxygen atom, a sulfur atom and a nitrogen atom as the ring-constituting atoms besides carbon atoms, such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl and the like; and a bicyclic or tricyclic aromatic fused heterocycle having 3 to 13 carbon atoms and containing 1 to 5 heteroatom(s) selected from an oxygen atom, a sulfur atom and a nitrogen atom as the ring-constituting atoms besides carbon atoms, such as benzofuranyl, isobenzofuranyl, benzo[b]thienyl, indolyl, isoindolyl, 1H-indazolyl, benzimidazolyl, benzoxazolyl, benzothiazolyl, 1H-benzotriazolyl, quinolyl, isoquinolyl, cinnolyl, quinazolyl, quinoxalinyl, phthalazinyl, naphthylidinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, indolidinyl, pyrrolo[1,2-b]pyridazinyl, pyrazolo[1,5-a]pyridyl, imidazo[1,2-a]pyridyl, imidazo[l,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like.

Preferable examples of the nonaromatic heterocycle group include those having 2 to 10 carbon atoms and containing 1 to 3 heteroatom(s) selected from an oxygen atom, a sulfur atom and a nitrogen atom as the ring-constituting atoms besides carbon atoms, such as oxiranyl, azetidinyl, oxetanyl, thietanyl, pyrrolidinyl, tetrahydrofuryl, tetrahydropyranyl, morpholinyl, thiomorpholinyl, piperazinyl, pyrrolidinyl, piperidino, morpholino, thiomorpholino and the like.

Examples of the halogen atom include fluorine, chlorine, bromine and iodine. Among them, fluorine and chlorine are preferred.

The optionally substituted amino group includes an amino group optionally mono- or di-substituted with an alkyl group having 1 to 10 carbon atom(s), a cycloalkyl group having 3 to 10 carbon atoms, an alkenyl group having 2 to 10 carbon atoms, a cycloalkenyl group having 3 to 10 carbon atoms, an acyl group having 1 to 13 carbon atom(s) (e.g., an alkanoyl group having 2 to 10 carbon atoms, an arylcarbonyl group having 7 to 13 carbon atoms and the like) or an aryl group having 6 to 12 carbon atoms and the like. The acyl group as referred herein is as defined for the below-mentioned optionally substituted acyl group.

The substituted amino group includes methylamino, dimethylamino, ethylamino, diethylamino, propylamino, dibutylamino, diallylamino, cyclohexylamino, acetylamino, propionylamino, benzoylamino, phenylamino, N-methyl-N-phenylamino and the like.

The acyl group of the optionally substituted acyl group includes an acyl group having 1 to 13 carbon atom(s), specifically formyl, as well as a group in which a carbonyl group is linked with an alkyl group having 1 to 10 carbon atom(s), a cycloalkyl group having 3 to 10 carbon atoms, an alkenyl group having 2 to 10 carbon atoms, a cycloalkenyl group having 3 to 10 carbon atoms, an aryl group having 6 to 12 carbon atoms or an aromatic heterocycle group (e.g., thienyl, furyl, pyridyl and the like) and the like.

Preferable examples of the acyl group include acetyl, propionyl, butyryl, isobutyryl, valeryl, isovaleryl, pivaloyl, hexanoyl, heptanoyl, octanoyl, cyclobutanecarbonyl, cyclopentanecarbonyl, cyclohexanecarbonyl, cycloheptanecarbonyl, crotonyl, 2-cyclohexenecarbonyl, benzoyl, nicotinoyl, isonicotinoyl and the like.

The acyl group may have 1 to 3 substituent(s) at the substitutable position(s), and such substituents include an alkyl group having 1 to 3 carbon atom(s), an alkoxy group having 1 to 3 carbon atom(s), a halogen (e.g., fluorine, chlorine, iodine and the like), nitro, hydroxy, amino and the like.

Other forms of the acyl group are represented by the following general formula:

—COR¹¹, —SO₂R¹⁴, —SOR¹⁵ or —PO₃R¹⁶R¹⁷

wherein R¹¹, R¹⁴, R¹⁵, R¹⁶ and R¹⁷ may be the same or different and each is an optionally substituted hydrocarbon group.

Examples of the hydrocarbon group of the “optionally substituted hydrocarbon group” represented by R¹¹, R¹⁴ , R¹⁵, R¹⁶ and R¹⁷ include an alkyl group having 1 to 10 carbon atom(s), a cycloalkyl group having 3 to 10 carbon atoms, an alkenyl group having 2 to 10 carbon atoms, a cycloalkenyl group having 3 to 10 carbon atoms and an aryl group having 6 to 12 carbon atoms. Furthermore, the substituents for the above-mentioned “optionally substituted hydrocarbon group” include a C₁₋₆ alkyl group (when the hydrocarbon group is other than an alkyl group), a C₁₋₆ alkoxy group, a halogen atom (e.g., fluorine, chlorine, bromine, iodine and the like), a nitro group, a C₁₋₆ haloalkyl group and a C₁₋₆ haloalkoxy group. The number of the substituents is, for example, 1 to 3.

For the optionally substituted hydroxy group, the substituted hydroxy group includes an alkoxy group, an alkenyloxy group, an aralkyloxy group, an acyloxy group and an aryloxy group, each of which is optionally substituted, and the like.

Preferable examples of the alkoxy group include an alkoxy group having 1 to 10 carbon atom(s) such as methoxy, ethoxy, propoxy, isopropoxy, butoxy, isobutoxy, sec.-butoxy, t.-butoxy, pentyloxy, isopentyloxy, neopentyloxy, hexyloxy, heptyloxy, nonyloxy, cyclobutoxy, cyclopentyloxy, cyclohexyloxy and the like.

Preferable examples of the alkenyloxy group include an alkenyloxy group having 2 to 10 carbon atoms such as allyloxy, crotyloxy, 2-pentenyloxy, 3-hexenyloxy, 2-cyclopentenylmethoxy, 2-cyclohexenylmethoxy and the like.

Preferable examples of the aralkyloxy group include an aralkyloxy group having 7 to 10 carbon atoms such as a phenyl-C₁₋₄ alkyloxy (e.g., benzyloxy, phenethyloxy and the like) and the like.

Preferable examples of the acyloxy group include an acyloxy group having 2 to 13 carbon atoms, more preferably alkanoyloxy having 2 to 4 carbon atoms (e.g., acetyloxy, propionyloxy, butyryloxy, isobutyryloxy and the like) and the like.

Preferable examples of the aryloxy group include an aryloxy group having 6 to 14 carbon atoms such as phenoxy, naphthyloxy and the like.

The above-mentioned alkoxy group, alkenyloxy group, aralkyloxy group, acyloxy group and aryloxy group may have 1 or 2 substituent(s) at the substitutable position(s), and such substituents include a halogen (e.g., fluorine, chlorine, bromine and the like), an alkoxy group having 1 to 3 carbon atom(s) and the like. For example, the substituted aryloxy group includes 4-chlorophenoxy, 2-methoxyphenoxy and the like.

For the optionally substituted thiol group, the substituted thiol group includes alkylthio, cycloalkylthio, aralkylthio, acylthio, arylthio, heteroarylthio and the like.

Preferable examples of the alkylthio group include an alkylthio group having 1 to 10 carbon atom(s) such as methylthio, ethylthio, propylthio, isopropylthio, butylthio, isobutylthio, sec.-butylthio, t.-butylthio, pentylthio, isopentylthio, neopentylthio, hexylthio, heptylthio, nonylthio and the like.

Preferable examples of the cycloalkylthio group include a cycloalkylthio group having 3 to 10 carbon atoms such as cyclobutylthio, cyclopentylthio, cyclohexylthio and the like.

Preferable examples of the aralkylthio group include an aralkylthio group having 7 to 10 carbon atoms such as a phenyl-C₁₋₄ alkylthio (e.g., benzylthio, phenethylthio and the like) and the like.

Preferable examples of the acylthio group include an acylthio group having 2 to 13 carbon atoms, more preferably an alkanoylthio group having 2 to 4 carbon atoms (e.g., acetylthio, propionylthio, butyrylthio, isobutyrylthio and the like) and the like.

Preferable examples of the arylthio group include an arylthio group having 6 to 14 carbon atoms such as phenylthio, naphthylthio and the like.

Preferable examples of the heteroarylthio group include 2-pyridylthio, 3-pyridylthio, as well as 2-imidazolylthio, 1,2,4-triazol-5-ylthio and the like.

The above-mentioned alkylthio group, cycloalkylthio group, aralkylthio group, acylthio group and arylthio group, and heteroarylthio group may have 1 or 2 substituent(s) at the substitutable position(s), and such substituents include a halogen (e.g., fluorine, chlorine, bromine and the like), an alkoxy group having 1 to 3 carbon atom(s) and the like.

For the optionally esterified carboxyl group, the esterified carboxyl group includes an alkoxycarbonyl group having 2 to 5 carbon atoms (e.g., methoxycarbonyl, ethoxycarbonyl, propoxycarbonyl, butoxycarbonyl and the like), an aralkyloxycarbonyl group having 8 to 10 carbon atoms (e.g., benzyloxycarbonyl and the like), an aryloxycarbonyl group having 7 to 15 carbon atoms optionally substituted with 1 or 2 alkyl group(s) having 1 to 3 carbon atom(s) (e.g., phenoxycarbonyl, p-tolyloxycarbonyl and the like) and the like.

For the optionally amidated carboxyl group, the amidated-carboxyl group includes a group represented by the formula:

—CON(R¹²)(R¹³)

wherein R¹² and R¹³ may be the same or different and each is a hydrogen atom, an optionally substituted hydrocarbon group or an optionally substituted heterocycle group.

Here, the hydrocarbon group of the “optionally substituted hydrocarbon group” and the heterocycle group of the “optionally substituted heterocycle group” each of which is represented by R¹² and R¹³ include the above-mentioned aliphatic hydrocarbon groups, alicyclic hydrocarbon groups, aromatic hydrocarbon groups and heterocycle groups which are exemplified hereinbefore as the hydrocarbon group of the “optionally substituted hydrocarbon group” represented by R¹ and the heterocycle group of the “optionally substituted heterocycle group” represented by R¹, respectively. The hydrocarbon group and heterocycle group may have 1 to 3 substituent(s) at the substitutable position(s), and such substituents include a halogen (e.g., fluorine, chlorine, bromine, iodine and the like), an alkyl group having 1 to 4 carbon atom(s), an alkoxy group having 1 to 4 carbon atom(s) and the like.

Substituents for the hydrocarbon group or heterocycle group represented by R¹ are preferably an alkyl group having 1 to 10 carbon atom(s), an aromatic heterocycle group, an aryl group having 6 to 14 carbon atoms, and more preferably an alkyl having 1 to 3 carbon atom(s), furyl, thienyl, phenyl or naphthyl.

When the hydrocarbon group or heterocycle group represented by R¹ is an alicyclic hydrocarbon group, an aromatic hydrocarbon group, an aromatic heterocycle group or a nonaromatic heterocycle group, it may further have one or more, preferably 1 to 3 of suitable substituent(s), and such substituents include an alkyl group having 1 to 6 carbon atom(s), an alkenyl group having 2 to 6 carbon atoms, a cycloalkyl group having 3 to 10 carbon atoms, an aryl group having 6 to 14 carbon atoms (e.g., phenyl, naphthyl and the like), an aromatic heterocycle group (e.g., thienyl, furyl, pyridyl, oxazolyl, thiazolyl and the like), a nonaromatic heterocycle group (e.g., tetrahydrofuryl, morpholino, thiomorpholino, piperidino, pyrrolidinyl, piperazinyl and the like), an aralkyl group having 7 to 9 carbon atoms, an amino group, an amino group mono- or di-substituted with an alkyl group having 1 to 4 carbon atom(s) or an acyl group having 2 to 8 carbon atoms (e.g., an alkanoyl group and the like), an amidino group, an acyl group having 2 to 8 carbon atoms (e.g., an alkanoyl group and the like), a carbamoyl group, a carbamoyl group mono or di-substituted with an alkyl group having 1 to 4 carbon atom(s), sulfamoyl group, a sulfamoyl group mono- or di-substituted with an alkyl group having 1 to 4 carbon atom(s), carboxyl group, an alkoxycarbonyl group having 2 to 8 carbon atoms, a hydroxy group, an alkoxy group having 1 to 6 carbon atom(s), an alkenyloxy group having 2 to 5 carbon atoms, a cycloalkyloxy group having 3 to 7 carbon atoms, an aralkyloxy group having 7 to 9 carbon atoms, an aryloxy group having 6 to 14 carbon atoms (e.g., phenyloxy, naphthyloxy and the like), a thiol group, an alkylthio group having 1 to 6 carbon atom(s), an aralkylthio group having 7 to 9 carbon atoms, an arylthio group having 6 to 14 carbon atoms (e.g., phenylthio, naphthylthio and the like), a sulfo group, a cyano group, an azido group, a nitro group, a nitroso group, a halogen atom (e.g., fluorine, chlorine, bromine, iodine) and the like.

R¹ is preferably an optionally substituted heterocycle group, and more preferably pyridyl, oxazolyl, thiazolyl or triazolyl, each of which is optionally substituted. R¹ is specifically preferably pyridyl, oxazolyl, thiazolyl or triazolyl, each of which optionally have 1 or 2 substituents selected form alkyl having 1 to 3 carbon atom(s), cycloalkyl having 3 to 7 carbon atoms, furyl, thienyl, phenyl and naphthyl. Here, the furyl, thienyl, phenyl and naphthyl may have 1 or 2 of alkyl having 1 to 3 carbon atom(s), alkoxy having 1 to 3 carbon atom(s), a halogen (e.g., fluorine, chlorine, bromine, iodine and the like) or haloalkyl having 1 to 3 carbon atom(s) as a substituent.

Preferable examples of R¹ include an optionally substituted heterocycle group or optionally substituted cyclic hydrocarbon group represented by the following formulas:

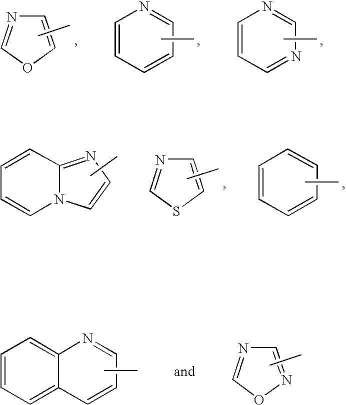

These substituents may have 1 or 2 substituent(s) selected from phenyl, furyl, thienyl and alkyl having 1 to 4 carbon atom(s). The phenyl, furyl and thienyl may have 1 or 2 substituent(s) selected from alkyl having 1 to 6 carbon atom(s), alkoxy having 1 to 6 carbon atom(s), a halogen (e.g., fluorine, chlorine, bromine, iodine and the like), nitro, haloalkyl having 1 to 6 carbon atom(s) and haloalkoxy having 1 to 6 carbon atom(s). Furthermore, the alkyl having 1 to 4 carbon atom(s) may have 1 or 2 substituent(s) selected from alkoxy having 1 to 6 carbon atom(s), a halogen (e.g., fluorine, chlorine, bromine, iodine and the like), nitro, haloalkyl having 1 to 6 carbon atom(s) and haloalkoxy having 1 to 6 carbon atom(s).

R¹ is more preferably a group represented by the following formula:

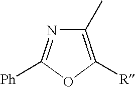

wherein Ph is an optionally substituted phenyl group, R″ is a hydrogen atom or an optionally substituted alkyl group having 1 to 6 carbon atom(s).

Substituents for the phenyl group represented by Ph and an alkyl group having 1 to 6 carbon atom(s) represented by R″ include alkoxy having 1 to 6 carbon atom(s), a halogen (e.g., fluorine, chlorine, bromine, iodine and the like), nitro, haloalkyl having 1 to 6 carbon atom(s) and haloalkoxy having 1 to 6 carbon atom(s). The number of the substituents is, for example, 1 to 3.

X is a bond, —CO—, —CH(OH)— or a group represented by —NR⁶—, wherein R⁶ is a hydrogen atom or an optionally substituted alkyl group, preferably a bond, —CH(OH)— or —NR⁶— and more preferably a bond or —NR⁶—.

Here, the alkyl group of the “optionally substituted alkyl group” represented by R⁶ includes an alkyl group having 1 to 4 carbon atom(s) such as methyl, ethyl, propyl, isopropyl, butyl, isobutyl, sec.-butyl, t.-butyl and the like. The alkyl group may have 1 to 3 substituent(s) at the substitutable position(s), and such substituents include a halogen (e.g., fluorine, chlorine, bromine, iodine), an alkoxy group having 1 to 4 carbon atom(s) (e.g., methoxy, ethoxy, propoxy, isopropoxy, butoxy, isobutoxy, sec.-butoxy, t.-butoxy and the like), a hydroxy group, a nitro group, and an acyl group having 1 to 4 carbon atom(s) (e.g., an alkanoyl group having 1 to 4 carbon atom(s) such as formyl, acetyl and propionyl).

n is an integer of 1 to 3, preferably 1 or 2.

Y is an oxygen atom, a sulfur atom, —SO—, —SO₂— or —NR⁷—, wherein R⁷ is a hydrogen atom or an optionally substituted alkyl group, preferably an oxygen atom, a sulfur atom or —NR⁷—. Here, the “optionally substituted alkyl group” represented by R⁷ includes those similar to the above-mentioned “optionally substituted alkyl group” represented by the R⁶.

The ring A is a benzene ring, and the benzene ring may have further 1 to 3 substituent(s) at the substitutable position(s). Such substituents include an alkyl group, an optionally substituted hydroxy group, a halogen atom, an optionally substituted acyl group, a nitro group, and an optionally substituted amino group, and these groups are similar to those exemplified as the substituents for the hydrocarbon group and heterocycle group represented by R¹.

The substituent is preferably an alkyl group having 1 to 4 carbon atom(s), an alkoxy group having 1 to 4 carbon atom(s) or a halogen atom.

Furthermore, in the general formula (I) the partial structural formula:

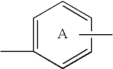

is preferably

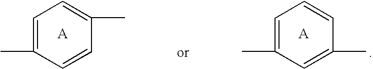

p is an integer of 1 to 8, preferably an integer of 1 to 3.

The “optionally substituted hydrocarbon group” represented by R² includes those exemplified as the “optionally substituted hydrocarbon group” represented by R¹.

The “optionally substituted heterocycle group” represented by R² includes those exemplified as the “optionally substituted heterocycle group” represented by R¹.

R² is preferably an optionally substituted hydrocarbon group. R² is more preferably an aliphatic hydrocarbon group, an alicyclic hydrocarbon group, an aromatic aliphatic hydrocarbon group or an aromatic hydrocarbon group, each of which is optionally substituted, and more preferably an alkyl group having 1 to 4 carbon atom(s), a phenylalkenyl group having 8 to 10 carbon atoms or an aryl group having 6 to 14 carbon atoms, each of which is optionally substituted.

Substituent which the hydrocarbon group may have is preferably a halogen atom, an alkoxy group having 1 to 4 carbon atom(s), an aryloxy group having 6 to 14 carbon atoms and an aromatic heterocycle group (e.g., furyl, thienyl). The number of the substituents is, for example, 1 to 3.

q is an integer of 0 to 6, preferably 0 to 4. m is 0 or 1.

R³ is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an optionally substituted acyl group, or R⁹ and R¹⁰ may be linked to form a ring.

The “optionally substituted hydrocarbon group” represented by R⁸ includes those exemplified as the “optionally substituted hydrocarbon group” represented by R¹. Preferably, R⁸ is “an alkyl group having 1 to 4 carbon atom(s)” and “an aryl group having 6 to 10 carbon atoms optionally substituted with an alkyl group having 1 to 4 carbon atom(s) or a halogen atom”. Here, the above-mentioned alkyl group having 1 to 4 carbon atom(s) includes methyl, ethyl, propyl, butyl, isobutyl, sec-butyl and t-butyl. Among them, methyl and ethyl are preferred. A halogen for the “aryl group having 6 to 10 carbon atoms optionally substituted with an alkyl group having 1 to 4 carbon atom(s) or a halogen atom” includes fluorine, chlorine, bromine and iodine. Among them, chlorine is preferable. The aryl group having 6 to 10 carbon atoms includes phenyl and naphthyl. Among them, phenyl is preferred.

The “optionally substituted hydrocarbon group” and “optionally substituted heterocycle group” represented by R⁹ and R¹⁰ include those similar to the “optionally substituted hydrocarbon group” and “optionally substituted heterocycle group” represented by R¹, respectively.

The “optionally substituted acyl group” represented by R⁹ and R¹⁰ includes those similar to the “optionally substituted acyl group” exemplified as substituents which the “optionally substituted hydrocarbon group” represented by R¹ may have.

R⁹ and R¹⁰ may be linked to form a 5- to 7-membered cyclic amino group, and specific cyclic amino group includes 1-pyrrolidinyl, 1-piperidinyl, 1-hexamethyleneiminyl, 4-morpholino, 4-thiomorpholino and the like.

R⁴ and R⁵ may be the same or different and each is a hydrogen atom or an optionally substituted hydrocarbon group, or R⁴ and R² may be linked to form a ring.

The “optionally substituted hydrocarbon group” represented by R⁴ and R⁵ includes those similar to the “optionally substituted hydrocarbon group” exemplified as the above-mentioned R¹, and preferably includes those similar to the “optionally substituted alkyl group” represented by the above-mentioned R⁶ and the like.

R⁴ and R² may be linked to form a ring. The ring formed by linking of R⁴ and R² includes cycloalkane having 5 to 11 carbon atoms and cycloalkene having 5 to 11 carbon atoms, specifically cyclopentane, cyclopentene, cyclohexane, cyclohexene, cycloheptane, cycloheptene, cyclooctane, cyclooctene, cyclononane, cyclononene, cyclodecane, cyclodecene, cycloundecane and cycloundecene and the like.

For compound (I), there are an (E)-form and a (Z)-form regarding the imino bond. The compound includes the (E)-form and (Z)-form by itself, and a mixture thereof.

The compound (I) is preferably a compound represented by the following formula:

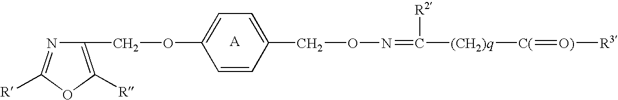

wherein R′ is a phenyl, furyl or thienyl group, each of which is optionally substituted with 1 or 2 substituent(s) selected from alkyl having 1 to 6 carbon atom(s), alkoxy having 1 to 6 carbon atom(s), a halogen (e.g., fluorine, chlorine, bromine, iodine and the like), nitro, haloalkyl having 1 to 6 carbon atom(s) and haloalkoxy having 1 to 6 carbon atom(s); R″ is a hydrogen atom or optionally substituted alkyl having 1 to 6 carbon atom(s) (more preferably a hydrogen atom, a methyl group or an ethyl group); R^{2′} is a phenyl group optionally substituted with at least one substituent(s) selected from the group consisting of a hydrogen atom, alkyl having 1 to 6 carbon atom(s), alkoxy having 1 to 6 carbon atom(s) and a halogen; q is an integer of 1 to 6; R^{3′} is hydroxy, alkoxy having 1 to 6 carbon atom(s), or a group represented by the formula —NR⁹R¹⁰, wherein R⁹ and R¹⁰ may be the same or different and each is a hydrogen atom, an optionally substituted hydrocarbon group, an optionally substituted heterocycle group or an optionally substituted acyl group, or R⁹ and R¹⁰ may be linked to form a ring; and ring A is an optionally substituted benzene ring.

The compound (I) is more preferably a compound represented by the following formula:

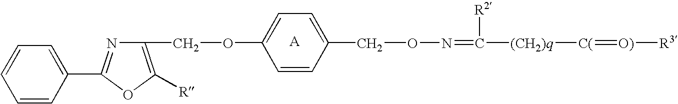

wherein each symbol is as defined above.

Preferred embodiments of compound (I) include the following compounds (1) to (10) and the like.

(1) Z-2-[4-(5-methyl-2-phenyl-4-oxazolylmethoxy)benzyloxyimino]-2-phenylacetic acid;

(2) Z-4-[4-(5-methyl-2-phenyl-4-oxazolylmethoxy)benzyloxyimino]-4-phenylbutyric acid;

(3) Z-2-(4-bromophenyl)-2-[4-(5-methyl-2-phenyl-4-oxazolylmethoxy)benzyloxyimino]acetic acid;

(4) Z-2-[4-(5-methyl-2-phenyl-4-oxazolylmethoxy)benzyloxyimino]-2-(4-phenoxyphenyl)acetic acid;

(5) Z-4-(4-fluorophenyl)-4-[4-(5-methyl-2-phenyl-4-oxazolylmethoxy)benzyloxyimino]butyric acid;

(6) Z-3-methyl-2-[4-(5-methyl-2-phenyl-4-oxazolylmethoxy)benzyloxyimino]butyric acid;

(7) E-4-[4-(5-methyl-2-phenyl-4-oxazolylmethoxy)benzyloxyimino]-4-phenylbutyric acid;

(8) E-4-(4-fluorophenyl)-4-[4-(5-methyl-2-phenyl-4-oxazolylmethoxy)benzyloxyimino]butyric acid;

(9) E-4-[4-(5-methyl-2-phenyl-4-oxazolylmethoxy)benzyloxyimino]-4-phenylbutyramide;

(10) E-8-[4-(5-methyl-2-phenyl-4-oxazolylmethoxy)benzyloxyimino]-8-phenyloctanoic acid.

Hereinafter these compounds are sometimes abbreviated as merely the compound (1), compound (2) and the like. Among these compounds, the compound (7) is specifically preferred.

A salt of the compound (I) includes salts similar to the above-mentioned salts of pioglitazone. Specifically, a sodium salt, potassium salt, hydrochloride and the like are preferred.

The compound (I) can be prepared, for example, according to the method disclosed in WO99/58510.

Hereinafter each symbol in the formula (II) is specifically explained.

The hydrocarbon group of the “optionally substituted hydrocarbon group” represented by R²¹ includes an aliphatic hydrocarbon group, an alicyclic hydrocarbon group, an alicyclic-aliphatic hydrocarbon group, an aromatic aliphatic hydrocarbon group and an aromatic hydrocarbon group. The number of carbon atoms contained in the hydrocarbon group is preferably 1 to 14.

The aliphatic hydrocarbon group is preferably an aliphatic hydrocarbon group having 1 to 8 carbon atom(s). The aliphatic hydrocarbon group includes a saturated aliphatic hydrocarbon group having 1 to 8 carbon atom(s) (e.g., an alkyl group and the like) such as methyl, ethyl, propyl, isopropyl, butyl, isobutyl, sec.-butyl, t.-butyl, pentyl, isopentyl, neopentyl, hexyl, isohexyl, heptyl, octyl and the like; an unsaturated aliphatic hydrocarbon group having 2 to 8 carbon atoms (e.g., an alkenyl group having 2 to 8 carbon atoms, an alkadienyl group having 4 to 8 carbon atoms, an alkenyl alkynyl group having 2 to 8 carbon atoms, an alkadiynyl group having 4 to 8 carbon atoms and the like) such as ethenyl, 1-propenyl, 2-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 3-hexenyl, 2,4-hexadienyl, 5-hexenyl, 1-heptenyl, 1-octenyl, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 2,4-hexadiynyl, 5-hexynyl, 1-heptynyl, 1-octynyl and the like.

The alicyclic hydrocarbon group is preferably an alicyclic hydrocarbon group having 3 to 7 carbon atoms. The alicyclic hydrocarbon group includes a saturated alicyclic hydrocarbon group having 3 to 7 carbon atoms (e.g., a cycloalkyl group and the like) such as cyclopropyl, cyclobutyl, cyclopentyl, cyclohexyl, cycloheptyl and the like; an unsaturated alicyclic hydrocarbon group having 5 to 7 carbon atoms (e.g., a cycloalkenyl group, a cycloalkadienyl group and the like) such as 1-cyclopentenyl, 2-cyclopentenyl, 3-cyclopentenyl, 1-cyclohexenyl, 2-cyclohexenyl, 3-cyclohexenyl, 1-cycloheptenyl, 2-cycloheptenyl, 3-cycloheptenyl, 2,4-cycloheptadienyl and the like.

The alicyclic-aliphatic hydrocarbon group includes groups obtained by linking the above-mentioned alicyclic hydrocarbon group and aliphatic hydrocarbon group (e.g., a cycloalkyl-alkyl group, a cycloalkenyl-alkyl group and the like). Among them, an alicyclic-aliphatic hydrocarbon group having 4 to 9 carbon atoms is preferred. The alicyclic-aliphatic hydrocarbon group includes cyclopropylmethyl, cyclopropylethyl, cyclobutylmethyl, cyclopentylmethyl, 2-cyclopentenylmethyl, 3-cyclopentenylmethyl, cyclohexylmethyl, 2-cyclohexenylmethyl, 3-cyclohexenylmethyl, cyclohexylethyl, cyclohexylpropyl, cycloheptylmethyl, cycloheptylethyl and the like.

The aromatic aliphatic hydrocarbon group is preferably an aromatic aliphatic hydrocarbon group having 7 to 13 carbon atoms (e.g., an aralkyl group having 7 to 13 carbon atoms, an arylalkenyl group having 8 to 13 carbon atoms and the like). The aromatic aliphatic hydrocarbon group include phenylalkyl having 7 to 9 carbon atoms such as benzyl, phenethyl, 1-phenylethyl, 1-phenylpropyl, 2-phenylpropyl, 3-phenylpropyl and the like; naphthylalkyl having 11 to 13 carbon atoms such as α-naphthylmethyl, α-naphthylethyl, β-naphthylmethyl, β-naphthylethyl and the like; phenylalkenyl having 8 to 10 carbon atoms such as styryl and the like; naphthylalkenyl having 12 or 13 carbon atoms such as 2-(2-naphthylvinyl) and the like.

The aromatic hydrocarbon group is preferably an aromatic hydrocarbon group having 6 to 14 carbon atoms (e.g., an aryl group and the like). The aromatic hydrocarbon group includes phenyl, naphthyl, anthryl, phenanthryl, acenaphthylenyl, biphenylyl and the like. Among them, phenyl, 1-naphthyl, 2-naphthyl and the like are preferred.

Among the above-mentioned hydrocarbon groups, a cyclic hydrocarbon group such as an alicyclic hydrocarbon group and an aromatic hydrocarbon group is preferred. The hydrocarbon group is more preferably an aromatic hydrocarbon group having 6 to 14 carbon atoms, and especially preferably phenyl or naphthyl.

The heterocycle group of the “optionally substituted heterocycle group” represented by R²¹ includes a 5- to 7-membered monocyclic heterocycle group containing 1 to 4 heteroatom(s) selected from an oxygen atom, a sulfur atom and a nitrogen atom as the ring-constituting atoms besides carbon atoms, or a fused heterocycle group. The fused heterocycle group includes a group in which the 5- to 7-membered monocyclic heterocycle group is fused with a 6-membered ring containing 1 or 2 nitrogen atom(s), a benzene ring or a 5-membered ring containing one sulfur atom, and the like.

Specific examples of the heterocycle group include an aromatic heterocycle group such as 2-furyl, 3-furyl, 2-thienyl, 3-thienyl, 2-pyridyl, 3-pyridyl, 4-pyridyl, 2-pyrimidinyl, 4-pyrimidinyl, 5-pyrimidinyl, 6-pyrimidinyl, 3-pyridazinyl, 4-pyridazinyl, 2-pyrazinyl, 1-pyrrolyl, 2-pyrrolyl, 3-pyrrolyl, 1-imidazolyl, 2-imidazolyl, 4-imidazolyl, 5-imidazolyl, 1-pyrazolyl, 3-pyrazolyl, 4-pyrazolyl, isoxazolyl, isothiazolyl, 2-thiazolyl, 4-thiazolyl, 5-thiazolyl, 2-oxazolyl, 4-oxazolyl, 5-oxazolyl, 1,2,4-oxadiazol-3-yl, 1,2,4-oxadiazol-5-yl, 1,3,4-oxadiazol-2-yl, 1,3,4-thiadiazol-2-yl, 1,2,4-triazol-1-yl, 1,2,4-triazol-3-yl, 1,2,3-triazol-1-yl, 1,2,3-triazol-2-yl, 1,2,3-triazol-4-yl, tetrazol-1-yl, tetrazol-5-yl, 2-quinolyl, 3-quinolyl, 4-quinolyl, 2-quinazolyl, 4-quinazolyl, 2-quinoxalyl, 2-benzoxazolyl, 2-benzothiazolyl, benzimidazol-1-yl, benzimidazol-2-yl, indol-1-yl, indol-3-yl, 1H-indazol-3-yl, 1H-pyrrolo[2,3-b]pyrazin-2-yl, 1H-pyrrolo[2,3-b]pyridin-6-yl, 1H-imidazo[4,5-b]pyridin-2-yl, 1H-imidazo[4,5-c]pyridin-2-yl, 1H-imidazo[4,5-b]pyrazin-2-yl, benzotriazol-1-yl and the like; and a nonaromatic heterocycle group such as 1-pyrrolidinyl, piperidino, morpholino, thiomorpholino, 1-piperazinyl, hexamethyleneimin-1-yl, oxazolidin-3-yl, thiazolidin-3-yl, imidazolidin-3-yl, 2-oxoimidazolidin-1-yl, 2,4-dioxoimidazolidin-3-yl, 2,4-dioxooxazolidin-3-yl, 2,4-dioxothiazolidin-3-yl, 1-oxo-phthalazin-2-yl, 2-oxo-2,3-dihydro-4H-1,4-benzothiazin-4-yl and the like.

The heterocycle group is preferably an aromatic heterocycle group, more preferably a 5- or 6-membered aromatic heterocycle group (preferably furyl, thienyl, pyridyl, pyrimidinyl, pyrazinyl, pyrazolyl, oxazolyl, thiazolyl, triazolyl, oxadiazolyl) which is optionally fused with a benzene ring. Among them, furyl, thienyl, pyridyl, pyrimidinyl, pyrazinyl, pyrazolyl, oxazolyl, thiazolyl, oxadiazolyl, benzoxazolyl, benzothiazolyl, quinolyl and the like are preferred.

The hydrocarbon group and heterocycle group represented by R²¹ may have 1 to 5, preferably 1 to 3 substituent(s) at the substitutable position(s). Such substituents include “a halogen atom”, “a nitro group”, “an optionally substituted aliphatic hydrocarbon group”, “an optionally substituted alicyclic hydrocarbon group”, “an optionally substituted aromatic hydrocarbon group”, “an optionally substituted aromatic heterocycle group”, “an optionally substituted nonaromatic heterocycle group”, “an optionally substituted acyl group”, “an optionally substituted amino group”, “an optionally substituted hydroxy group”, “an optionally substituted thiol group”, “an optionally esterified or amidated carboxyl group” and the like.

The “halogen atom” includes fluorine, chlorine, bromine and iodine. Among them, fluorine and chlorine are preferred.

The aliphatic hydrocarbon group of the “optionally substituted aliphatic hydrocarbon group” includes a straight or branched chain aliphatic hydrocarbon group having 1 to 15 carbon atom(s) such as an alkyl group, an alkenyl group, an alkynyl group and the like.

Preferable examples of the alkyl group include an alkyl group having 1 to 10 carbon atom(s) such as methyl, ethyl, propyl, isopropyl, butyl, isobutyl, sec.-butyl, t.-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like.

Preferable examples of the alkenyl group include an alkenyl group having 2 to 10 carbon atoms such as ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like.

Preferable examples of the alkynyl group include an alkynyl group having 2 to 10 carbon atoms such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like.

The substituents for the “optionally substituted aliphatic hydrocarbon group” include a cycloalkyl group having 3 to 10 carbon atoms, an aryl group having 6 to 14 carbon atoms (e.g., phenyl, naphthyl and the like), an aromatic heterocycle group (e.g., thienyl, furyl, pyridyl, oxazolyl, thiazolyl and the like), a nonaromatic heterocycle group (e.g., tetrahydrofuryl, morpholino, thiomorpholino, piperidino, pyrrolidinyl, piperazinyl and the like), an aralkyl group having 7 to 9 carbon atoms, an amino group, an amino group mono- or di-substituted with an alkyl group having 1 to 4 carbon atom(s) or an acyl group having 2 to 8 carbon atoms (e.g., an alkanoyl group and the like), an amidino group, an acyl group having 2 to 8 carbon atoms (e.g., an alkanoyl group and the like), a carbamoyl group, a carbamoyl group mono- or di-substituted with an alkyl group having 1 to 4 carbon atom(s), a sulfamoyl group, a sulfamoyl group mono- or di-substituted with an alkyl group having 1 to 4 carbon atom(s), a carboxyl group, an alkoxycarbonyl group having 2 to 8 carbon atoms, a hydroxy group, an alkoxy group having 1 to 6 carbon atom(s) optionally substituted with 1 to 3 halogen atom(s) (e.g., fluorine, chlorine, bromine, iodine and the like), an alkenyloxy group having 2 to 5 carbon atoms optionally substituted with 1 to 3 halogen atom(s) (e.g., fluorine, chlorine, bromine, iodine and the like), a cycloalkyloxy group having 3 to 7 carbon atoms, an aralkyloxy group having 7 to 9 carbon atoms, an aryloxy group having 6 to 14 carbon atoms (e.g., phenyloxy, naphthyloxy and the like), a thiol group, an alkylthio group having 1 to 6 carbon atom(s) optionally substituted with 1 to 3 halogen atom(s) (e.g., fluorine, chlorine, bromine, iodine and the like), an aralkylthio group having 7 to 9 carbon atoms, an arylthio group having 6 to 14 carbon atoms (e.g., phenylthio, naphthylthio and the like), a sulfo group, a cyano group, an azido group, a nitro group, a nitroso group, a halogen atom (e.g., fluorine, chlorine, bromine, iodine) and the like. The number of the substituents is, for example, 1 to 3.

The alicyclic hydrocarbon group of the “optionally substituted alicyclic hydrocarbon group” includes a saturated or unsaturated alicyclic hydrocarbon group having 3 to 12 carbon atoms such as a cycloalkyl group, a cycloalkenyl group, a cycloalkadienyl group and the like.

Preferable examples of the cycloalkyl group include a cycloalkyl group having 3 to 10 carbon atoms such as cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl and the like.

Preferable examples of the cycloalkenyl group include a cycloalkenyl group having 3 to 10 carbon atoms such as 2-cyclopenten-1-yl, 3-cyclopenten-1-yl, 2-cyclohexen-1-yl, 3-cyclohexen-1-yl and the like.

Preferable examples of the cycloalkadienyl group include a cycloalkadienyl group having 4 to 10 carbon atoms such as 2,4-cyclopentadien-1-yl, 2,4-cyclohexadien-1-yl, 2,5-cyclohexadien-1-yl and the like.

Preferable examples of the aromatic hydrocarbon group of the “optionally substituted aromatic hydrocarbon group” include an aromatic hydrocarbon group having 6 to 14 carbon atoms (e.g., an aryl group and the like) such as phenyl, naphthyl, anthryl, phenanthryl, acenaphthylenyl, biphenylyl and the like. Among them, phenyl, 1-naphthyl, 2-naphthyl and the like are preferred.

Preferable examples of the aromatic heterocycle group of the “optionally substituted aromatic heterocycle group” include a 5- to 7-membered aromatic monocyclic heterocycle group containing 1 to 4 heteroatom(s) selected from an oxygen atom, a sulfur atom and a nitrogen atom as the ring-constituting atoms besides carbon atoms, such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyrimidinyl, pyridazinyl, pyrazinyl, triazinyl and the like; and a bicyclic or tricyclic aromatic fused heterocycle having 3 to 13 carbon atoms and containing 1 to 5 heteroatom(s) selected from an oxygen atom, a sulfur atom and a nitrogen atom as the ring-constituting atoms besides carbon atoms, such as benzofuranyl, isobenzofuranyl, benzo[b]thienyl, indolyl, isoindolyl, 1H-indazolyl, benzimidazolyl, benzoxazolyl, benzothiazolyl, 1H-benzotriazolyl, quinolyl, isoquinolyl, cinnolyl, quinazolyl, quinoxalinyl, phthalazinyl, naphthylidinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, indolidinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like.

Preferable examples of the nonaromatic heterocycle group of the “optionally substituted nonaromatic heterocycle group” include a nonaromatic heterocycle group having 2 to 10 carbon atoms and containing 1 to 3 heteroatom(s) selected from an oxygen atom, a sulfur atom and a nitrogen atom as the ring-constituting atoms besides carbon atom, such as oxiranyl, azetidinyl, oxetanyl, thietanyl, pyrrolidinyl, tetrahydrofuryl, tetrahydropyranyl, morpholinyl, thiomorpholinyl, piperazinyl, pyrrolidinyl, piperidino, morpholino, thiomorpholino and the like.

The substituents for the above-mentioned “optionally substituted alicyclic hydrocarbon group”, “optionally substituted aromatic hydrocarbon group”, “optionally substituted aromatic heterocycle group” and “optionally substituted nonaromatic heterocycle group” include an alkyl group having 1 to 6 carbon atom(s) optionally substituted with 1 to 3 halogen atom(s) (e.g., fluorine, chlorine, bromine, iodine and the like), an alkenyl group having 2 to 6 carbon atoms optionally substituted with 1 to 3 halogen atom(s) (e.g., fluorine, chlorine, bromine, iodine and the like), a cycloalkyl group having 3 to 10 carbon atoms, an aryl group having 6 to 14 carbon atoms (e.g., phenyl, naphthyl and the like), an aromatic heterocycle group (e.g., thienyl, furyl, pyridyl, oxazolyl, thiazolyl and the like), a nonaromatic heterocycle group (e.g., tetrahydrofuryl, morpholino, thiomorpholino, piperidino, pyrrolidinyl, piperazinyl and the like), an aralkyl group having 7 to 9 carbon atoms, an amino group, an amino group mono- or di-substituted with an alkyl group having 1 to 4 carbon atom(s) or an acyl group having 2 to 8 carbon atoms (e.g., an alkanoyl group and the like), an amidino group, an acyl group having 2 to 8 carbon atoms (e.g., an alkanoyl group and the like), a carbamoyl group, a carbamoyl group monoor di-substituted with an alkyl group having 1 to 4 carbon atom(s), a sulfamoyl group, a sulfamoyl group mono- or di-substituted with an alkyl group having 1 to 4 carbon atom(s), a carboxyl group, an alkoxycarbonyl group having 2 to 8 carbon atoms, a hydroxy group, an alkoxy group having 1 to 6 carbon atom(s) optionally substituted with 1 to 3 halogen atom(s) (e.g., fluorine, chlorine, bromine, iodine and the like), an alkenyloxy group having 2 to 5 carbon atoms optionally substituted with 1 to 3 halogen atom(s) (e.g., fluorine, chlorine, bromine, iodine and the like), a cycloalkyloxy group having 3 to 7 carbon atoms, an aralkyloxy group having 7 to 9 carbon atoms, an aryloxy group having 6 to 14 carbon atoms (e.g., phenyloxy, naphthyloxy and the like), a thiol group, an alkylthio group having 1 to 6 carbon atom(s) optionally substituted with 1 to 3 halogen atom(s) (e.g., fluorine, chlorine, bromine, iodine and the like), an aralkylthio group having 7 to 9 carbon atoms, an arylthio group having 6 to 14 carbon atoms (e.g., phenylthio, naphthylthio and the like), a sulfo group, a cyano group, an azido group, a nitro group, a nitroso group, a halogen atom (e.g., fluorine, chlorine, bromine, iodine) and the like. The number of the substituents is, for example, 1 to 3.

The acyl group of the “optionally substituted acyl group” includes an acyl group having 1 to 13 carbon atom(s), specifically formyl, as well as a group represented by the formula: —COR³¹, —SO₂R³¹, —SOR³¹ or —PO₃R³¹R³², wherein R³¹ and R³² may be the same or different and each is a hydrocarbon group or an aromatic heterocycle group, and the like.

The hydrocarbon group represented by R³¹ or R³² includes the hydrocarbon groups exemplified for the above-mentioned R²¹. Among them, an alkyl group having 1 to 10 carbon atom(s), a cycloalkyl group having 3 to 10 carbon atoms, an alkenyl group having 2 to 10 carbon atoms, a cycloalkenyl group having 3 to 10 carbon atoms, an aryl group having 6 to 12 carbon atoms are preferred.

The aromatic heterocycle group represented by R³¹ or R³² includes the aromatic heterocycle groups exemplified for the above-mentioned R²¹. Among them, thienyl, furyl, pyridyl and the like are preferred.

Preferable examples of the acyl group include acetyl, propionyl, butyryl, isobutyryl, valeryl, isovaleryl, pivaloyl, hexanoyl, heptanoyl, octanoyl, cyclobutanecarbonyl, cyclopentanecarbonyl, cyclohexanecarbonyl, cycloheptanecarbonyl, crotonyl, 2-cyclohexenecarbonyl, benzoyl, nicotinoyl, isonicotinoyl and the like.

The acyl group may have 1 to 3 substituent(s) at the substitutable position(s), and such substituents include a C₁₋₆ alkyl group optionally substituted with 1 to 3 halogen atom(s) (e.g., fluorine, chlorine, iodine and the like), a C₁₋₆alkoxy group optionally substituted with 1 to 3 halogen atom(s) (e.g., fluorine, chlorine, bromine, iodine and the like), a halogen atom (e.g., fluorine, chlorine, bromine, iodine and the like), nitro, hydroxy, amino and the like.

The “optionally substituted amino group” includes an amino group optionally mono- or di-substituted with an alkyl group having 1 to 10 carbon atom(s), a cycloalkyl group having 3 to 10 carbon atoms, an alkenyl group having 2 to 10 carbon atoms, a cycloalkenyl group having 3 to 10 carbon atoms, an acyl group having 1 to 13 carbon atom(s) or an aryl group having 6 to 12 carbon atoms and the like.

Here, the acyl group includes those similar to the above-mentioned acyl groups and is preferably an alkanoyl group having 2 to 10 carbon atoms, an arylcarbonyl group having 7 to 13 carbon atoms or the like.

The substituted amino group includes methylamino, dimethylamino, ethylamino, diethylamino, propylamino, dibutylamino, diallylamino, cyclohexylamino, acetylamino, propionylamino, benzoylamino, phenylamino, N-methyl-N-phenylamino and the like.

The “optionally substituted hydroxy group” includes a hydroxy group optionally substituted with an alkyl group having 1 to 10 carbon atom(s), an alkenyl group having 2 to 10 carbon atoms, an aralkyl having 7 to 10 carbon atoms, an acyl group having 1 to 13 carbon atom(s) or an aryl group having 6 to 12 carbon atoms, each of which is optionally substituted.

Substituents which the “alkyl group having 1 to 10 carbon atom(s)”, “alkenyl group having 2 to 10 carbon atoms”, “aralkyl group having 7 to 10 carbon atoms”, “acyl group having 1 to 13 carbon atom(s)” and “aryl group having 6 to 12 carbon atoms” may have include a halogen atom (e.g., fluorine, chlorine, bromine, iodine and the like), a C₁₋₆ alkoxy group optionally substituted with 1 to 3 halogen atom(s) (e.g., fluorine, chlorine, bromine, iodine and the like), a hydroxy group, a nitro group, an amino group and the like. The number of the substituent is, for example, 1 or 2.

The substituted hydroxy group includes an alkoxy group, an alkenyloxy group, an aralkyloxy group, an acyloxy group, an aryloxy group and the like, each of which is optionally substituted.

Preferable examples of the alkoxy group include an alkoxy group having 1 to 10 carbon atom(s) such as methoxy, ethoxy, propoxy, isopropoxy, butoxy, isobutoxy, sec.-butoxy, t.-butoxy, pentyloxy, isopentyloxy, neopentyloxy, hexyloxy, heptyloxy, nonyloxy, cyclobutoxy, cyclopentyloxy, cyclohexyloxy and the like.

Preferable examples of the alkenyloxy group include an alkenyloxy group having 2 to 10 carbon atoms such as allyloxy, crotyloxy, 2-pentenyloxy, 3-hexenyloxy, 2-cyclopentenylmethoxy, 2-cyclohexenylmethoxy and the like.

Preferable examples of the aralkyloxy group include an aralkyloxy group having 7 to 10 carbon atoms such as phenyl-C₁₋₄ alkyloxy (e.g., benzyloxy, phenethyloxy and the like) and the like.

Preferable examples of the acyloxy group include an acyloxy group having 2 to 13 carbon atoms, more preferably an alkanoyloxy having 2 to 4 carbon atoms (e.g., acetyloxy, propionyloxy, butyryloxy, isobutyryloxy and the like) and the like.

Preferable examples of the aryloxy group include an aryloxy group having 6 to 14 carbon atoms such as phenoxy, naphthyloxy and the like.

The above-mentioned alkoxy group, alkenyloxy group, aralkyloxy group, acyloxy group and aryloxy group may have 1 or 2 substituent(s) at the substitutable position(s), and such substituents include a halogen atom (e.g.,.fluorine, chlorine, bromine, iodine and the like), a C₁₋₆ alkoxy group optionally substituted with 1 to 3 halogen atom(s) (e.g., fluorine, chlorine, bromine, iodine and the like), hydroxy, nitro, amino and the like. For example, the substituted aryloxy group is 4-chlorophenoxy, 2-methoxyphenoxy or the like.

The optionally substituted thiol group includes a thiol group optionally substituted with alkyl having 1 to 10 carbon atom(s), cycloalkyl having 3 to 10 carbon atoms, aralkyl having 7 to 10 carbon atoms, acyl having 2 to 13 carbon atoms, aryl having 6 to 14 carbon atoms, heteroaryl and the like.

The substituted thiol group includes alkylthio, cycloalkylthio, aralkylthio, acylthio, arylthio, heteroarylthio and the like.

Preferable examples of the alkylthio group include an alkylthio group having 1 to 10 carbon atom(s) such as methylthio, ethylthio, propylthio, isopropylthio, butylthio, isobutylthio, sec.-butylthio, t.-butylthio, pentylthio, isopentylthio, neopentylthio, hexylthio, heptylthio, nonylthio and the like.

Preferable examples of the cycloalkylthio group include a cycloalkylthio group having 3 to 10 carbon atoms such as cyclobutylthio, cyclopentylthio, cyclohexylthio and the like.

Preferable examples of the aralkylthio group include an aralkylthio group having 7 to 10 carbon atoms such as phenyl-C₁₋₄ alkylthio (e.g., benzylthio, phenethylthio and the like) and the like.

Preferable examples of the acylthio group include an acylthio group having 2 to 13 carbon atoms, more preferably an alkanoylthio group having 2 to 4 carbon atoms (e.g., acetylthio, propionylthio, butyrylthio, isobutyrylthio and the like) and the like.

Preferable examples of the arylthio group include an arylthio group having 6 to 14 carbon atoms such as phenylthio, naphthylthio and the like.

Preferable examples of the heteroarylthio group include 2-pyridylthio, 3-pyridylthio, as well as 2-imidazolylthio, 1,2,4-triazol-5-ylthio and the like.

For the optionally esterified carboxyl group, the esterified carboxyl group includes an alkoxycarbonyl group having 2 to 5 carbon atoms (e.g., methoxycarbonyl, ethoxycarbonyl, propoxycarbonyl, butoxycarbonyl and the like), an aralkyloxycarbonyl group having 8 to 10 carbon atoms (e.g., benzyloxycarbonyl and the like), an aryloxycarbonyl group having 7 to 15 carbon atoms optionally substituted with 1 or 2 alkyl group (s) having 1 to 3 carbon atom(s) (e.g., phenoxycarbonyl, p-tolyloxycarbonyl and the like) and the like.

For the optionally amidated carboxyl group, the amidated carboxyl group includes a group represented by the formula:

—CON(R³³)(R³⁴)

wherein R³³ and R³⁴ may be the same or different and each is a hydrogen atom, an optionally substituted hydrocarbon group or an optionally substituted heterocycle group.

Here, the hydrocarbon group of the “optionally substituted hydrocarbon group” represented by R³³ and R³⁴ includes the aliphatic hydrocarbon groups, alicyclic hydrocarbon groups and aromatic hydrocarbon groups exemplified as the hydrocarbon group of the “optionally substituted hydrocarbon group” represented by R²¹. Furthermore, the heterocycle group of the “optionally substituted heterocycle group” represented by R³³ and R³⁴ includes those exemplified as the heterocycle group of the “optionally substituted heterocycle group” represented by R²¹. The hydrocarbon group and heterocycle group may have 1 to 3 substituent(s) at the substitutable position(s), and such substituents include a halogen atom (e.g., fluorine, chlorine, bromine, iodine and the like), a C₁₋₆ alkyl group optionally substituted with 1 to 3 halogen atom(s) (e.g., fluorine, chlorine, bromine, iodine and the like), a C₁₋₆ alkoxy group optionally represented by 1 to 3 halogen atom(s) (e.g., fluorine, chlorine, bromine, iodine and the like), nitro, hydroxy, amino and the like.

Substituents for the hydrocarbon group and heterocycle group represented by R²¹ include preferably

1) an alkyl group having 1 to 10 (preferably 1 to 4) carbon atom(s) which optionally has 1 to 3 substituent(s) selected from an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino;

2) a cycloalkyl group having 3 to 10 (preferably 3 to 7) carbon atoms which optionally has 1 to 3 substituent(s) selected from an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino;

3) an aromatic heterocycle group (preferably furyl, thienyl, pyridyl, pyrazinyl and the like) which optionally has 1 to 3 substituent(s) selected from an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino;

4) an aryl group having 6 to 14 carbon atoms (preferably phenyl, naphthyl and the like) which optionally has 1 to 3 substituent(s) selected from an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino, and the like. The number of the substituent is, for example, 1 to 3, preferably 1 or 2.

The substituents are more preferably an alkyl group having 1 to 4 carbon atom(s), furyl, thienyl, phenyl, naphthyl and the like.

R²¹ is preferably an optionally substituted heterocycle group or an optionally substituted cyclic hydrocarbon group. R²¹ is more preferably an optionally substituted heterocycle group. Here, the heterocycle group is preferably a 5- or 6-membered aromatic heterocycle group (preferably furyl, thienyl, pyridyl, pyrimidinyl, pyrazinyl, oxazolyl, thiazolyl, triazolyl, oxadiazolyl, and pyrazolyl) which is optionally fused with a benzene ring. Among them, furyl, thienyl, pyridyl, pyrimidinyl, pyrazinyl, oxazolyl, thiazolyl, oxadiazolyl, benzoxazolyl, benzothiazolyl, quinolyl, pyrazolyl and the like are preferred.

Preferable examples of substituents, which the above-mentioned heterocycle group or cyclic hydrocarbon group may have, include 1) furyl, thienyl, pyridyl, pyrazinyl, phenyl or naphthyl, each of which optionally has 1 to 3 substituent(s) selected from.an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino; 2) an alkyl group having 1 to 4 carbon atom(s) or a cycloalkyl group having 3 to 7 carbon atoms, each of which optionally has 1 to 3 substituent(s) selected from an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino, and the like. The number of the substituent is, for example, 1 or 2.

R²¹ is specifically preferably pyridyl, oxazolyl, thiazolyl, triazolyl or pyrazolyl, each of which optionally has 1 or 2 substituent(s) selected from an alkyl group having 1 to 3 carbon atom(s), a cycloalkyl group having 3 to 7 carbon atoms, furyl, thienyl, pyridyl, phenyl and naphthyl.

Xa is a bond, an oxygen atom, a sulfur atom, —CO—, —CS—, —CR²⁴ (OR²⁵)— or —NR²⁶—, wherein R²⁴ and R²⁶ are each a hydrogen atom or an optionally substituted hydrocarbon group and R²⁵ is a hydrogen atom or a hydroxy protective group. Xa is preferably a bond, —CR²⁴(OR²⁵)— or —NR²⁶—, wherein each symbol is as defined above, and more preferably a bond or —NR²⁶— where the symbol is as defined above. Xa is specifically preferably a bond.

Here, the “optionally substituted hydrocarbon group” represented by R²⁴ and R²⁶ includes the “optionally substituted hydrocarbon group” exemplified for the above-mentioned R²¹. The “optionally substituted hydrocarbon group” is preferably an optionally substituted alkyl group having 1 to 4 carbon atom(s) such as methyl, ethyl, propyl, isopropyl, butyl, isobutyl, sec.-butyl, t.-butyl and the like. The alkyl group may have 1 to 3 substituent(s) at the substitutable position(s), and such substituents include a halogen atom (e.g., fluorine, chlorine, bromine, iodine), an alkoxy group having 1 to 4 carbon atom(s) (e.g., methoxy, ethoxy, propoxy, isopropoxy, butoxy, isobutoxy, sec.-butoxy, t.-butoxy and the like), hydroxy, nitro, amino, an acyl group having 1 to 4 carbon atom(s) (e.g., an alkanoyl group having 1 to 4 carbon atom(s) such as formyl, acetyl, propionyl and the like) and the like.

R²⁴ and R²⁶ are preferably a hydrogen atom or an alkyl group having 1 to 4 carbon atom(s).

The hydroxy protective group represented by R²⁵ includes C₁₋₆ alkyl (e.g., methyl, ethyl, propyl, isopropyl, butyl, tert-butyl and the like), phenyl, trityl, C₇₋₁₀ aralkyl (e.g., benzyl and the like), formyl, C₁₋₆ alkylcarbonyl (e.g., acetyl, propionyl and the like), benzoyl, C₇₋₁₀ aralkyl-carbonyl (e.g., benzylcarbonyl and the like), 2-tetrahydropyranyl, 2-tetrahydrofuranyl, silyl (e.g., trimethylsilyl, triethylsilyl, dimethylphenylsilyl, tert-butyldimethylsilyl, tert-butyldiethylsilyl and the like), C₂₋₆ alkenyl (e.g., 1-allyl and the like) and the like. These groups are optionally substituted with 1 to 3 halogen atom(s) (e.g., fluorine, chlorine, bromine, iodine and the like), C₁₋₆ alkyl (e.g., methyl, ethyl, propyl and the like), C₁₋₆ alkoxy (e.g., methoxy, ethoxy, propoxy and the like) or nitro and the like.

ma is an integer of 0 to 3, preferably an integer of 1 to 3, more preferably 1 or 2.

Ya is an oxygen atom, a sulfur atom, —SO—, —SO₂—, —NR²⁷—, —CONR²⁷— or —NR²⁷CO—, wherein R²⁷ is a hydrogen atom or an optionally substituted hydrocarbon group, and preferably an oxygen atom, a sulfur atom, —NR²⁷— or —NR²⁷CO—, wherein each symbol is as defined above.

Here, the “optionally substituted hydrocarbon group” represented by R²⁷ includes the “optionally substituted hydrocarbon group” exemplified for the above-mentioned R²⁴ and R²⁶. R²⁷ is preferably a hydrogen atom.

Ya is specifically preferably an oxygen atom.

The “aromatic ring” of the “aromatic ring optionally further having 1 to 3 substituent(s)” represented by ring Aa includes a benzene ring, a fused aromatic hydrocarbon ring, a 5- or 6-membered aromatic heterocycle, a fused aromatic heterocycle and the like.

Here, the “fused aromatic hydrocarbon ring” includes a fused aromatic hydrocarbon having 9 to 14 carbon atoms and the like. Specifically, it includes naphthalene, indene, fluorene, anthracene and the-like.

The “5- or 6-membered aromatic heterocycle” includes a 5- or 6-membered aromatic heterocycle containing 1 to 3 heteroatom(s) selected from a nitrogen atom, a sulfur atom and an oxygen atom besides carbon atoms, and the like. The specific examples include thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, furazan and the like.

The “fused aromatic heterocycle” includes a 9- or 14-membered (preferably 9- or 10-membered) fused aromatic heterocycle containing 1 to 4 heteroatom(s) selected from a nitrogen atom, a sulfur atom and an oxygen atom besides carbon atoms, and the like. The specific examples include benzofuran, benzothiophene, benzimidazole, benzoxazole, benzothiazole, benzisothiazole, naphtho[2,3-b]thiophene, isoquinoline, quinoline, indole, quinoxaline, phenanthridine, phenothiazine, phenoxazine, phthalazine, naphthylidine, quinazoline, cinnoline, carbazole, carboline, acridine, phenazine, phthalimide and the like.

The “aromatic ring” is preferably a benzene ring, a fused aromatic hydrocarbon ring having 9 to 14 carbon atoms (preferably naphthalene and the like), a 5- or 6-membered aromatic heterocycle (preferably pyridine, isoxazole and the like) or the like.

The “substituent” of the “aromatic ring optionally having further 1 to 3 substituent(s)” represented by ring Aa includes an optionally substituted aliphatic hydrocarbon group (preferably an alkyl group), an optionally substituted hydroxy group, a halogen atom, an optionally substituted acyl group, a nitro group, and an optionally substituted amino group and the like. As these substituents, those exemplified as the substituents for R¹ are used. Such substituents are preferably an alkyl group having 1 to 4 carbon atom(s), a hydroxy group, an alkoxy group having 1 to 4 carbon atom(s), an aralkyloxy group having 7 to 10 carbon atoms (preferably benzyloxy) and a halogen atom.

The ring Aa is preferably “a benzene or pyridine ring optionally further having 1 to 3 substituent(s)”, more preferably a benzene or pyridine ring optionally having 1 to 3 substituent(s) selected from an alkyl group having 1 to 4 carbon atom(s), a hydroxy group, an alkoxy group having 1 to 4 carbon atom(s), an aralkyloxy group having 7 to 10 carbon atoms and a halogen atom. The ring Aa is specifically preferably a benzene ring.

na is an integer of 1 to 8, preferably an integer of 1 to 3.

The “nitrogen-containing 5-membered heterocycle” of the “a nitrogen-containing 5-membered heterocycle optionally further substituted with alkyl group(s)” represented by ring B includes a 5-membered heterocycle containing at least one nitrogen atom as the ring-constituting atoms besides carbon atoms and optionally further containing 1 to 3 heteroatom(s) selected from an oxygen atom, a sulfur atom and a nitrogen atom.

Preferable examples of the nitrogen-containing 5-membered heterocycle include a 5-membered aromatic heterocycle such as pyrrole, pyrazole, imidazole, thiazole, isothiazole, oxazole, isoxazole, 1,2,4-triazole, 1,2,3-triazole, 1,2,3-oxadiazole, 1,2,4-oxadiazole, 1,3,4-oxadiazole, furazan, 1,2,3-thiadiazole, 1,2,4-thiadiazole, 1,3,4-thiadiazole, tetrazole and the like; and a 5-membered nonaromatic heterocycle such as pyrrolidine, imidazolidine, pyrazolidine and the like. The nitrogen-containing 5-membered heterocycle is preferably a 5-membered aromatic heterocycle containing at least one nitrogen atom as the ring-constituting atoms besides carbon atoms and optionally further containing one heteroatom selected from an oxygen atom, a sulfur atom and a nitrogen atom, such as pyrrole, pyrazole, imidazole, thiazole, isothiazole, oxazole, isoxazole and the like. Among them, pyrrole, pyrazole, imidazole and the like are preferred.

The “alkyl group” of the “nitrogen-containing 5-membered heterocycle optionally further substituted with alkyl group(s)” includes an alkyl group having 1 to 4 carbon atom(s). The specific examples include methyl, ethyl, propyl, butyl, isobutyl, sec-butyl, t-butyl and the like. Among them, methyl and ethyl are preferred.

The ring B is preferably pyrrole, pyrazole or imidazole, each of which is optionally further substituted with an alkyl group having 1 to 4 carbon atom(s). The ring B is specifically preferably pyrazole.

X¹ is a bond, an oxygen atom, a sulfur atom, —SO—, —SO₂—, —O—SO₂— or —NR³⁶—, wherein R³⁶ is a hydrogen atom or an optionally substituted hydrocarbon group.

Here, the “optionally substituted hydrocarbon group” represented by R³⁶ includes the “optionally substituted hydrocarbon group” exemplified for the above-mentioned R²⁴ and R²⁶.

X¹ is preferably a bond or an oxygen atom.

The “optionally substituted hydrocarbon group” and “optionally substituted heterocycle group” represented by R²² include the “optionally substituted hydrocarbon group” and “optionally substituted heterocycle group” exemplified for R²¹.

(1) When X¹ is a bond;

The hydrocarbon group of the “optionally substituted hydrocarbon group” represented by R²² is preferably an aliphatic hydrocarbon group (preferably an alkyl group) having 1 to 8 carbon atom(s), an aromatic hydrocarbon group having 6 to 14 carbon atoms and more preferably an aromatic hydrocarbon group having 6 to 14 carbon atoms (e.g., phenyl, naphthyl and the like).

The heterocycle group of the “optionally substituted heterocycle group” represented by R22 is preferably a 5- or 6-membered aromatic heterocycle group (e.g., furyl, thienyl, pyridyl and the like).

The substituents for the “optionally substituted hydrocarbon group” and “optionally substituted heterocycle group” are preferably 1) a halogen atom (e.g., fluorine, chlorine, bromine or iodine), 2) an alkyl group having 1 to 4 carbon atom(s) optionally substituted with 1 to 3 halogen atom(s) (e.g., fluorine, chlorine, bromine or iodine) (e.g., methyl, trifluoromethyl, propyl or isopropyl), 3) an alkoxy group having 1 to 4 carbon atom(s) optionally substituted with 1 to 3 halogen atom(s) (e.g., fluorine, chlorine, bromine or iodine) (e.g., methoxy or trifluoromethoxy), 4) an aralkyloxy group having 7 to 10 carbon atoms (e.g., benzyloxy), 5) an aryloxy group having 6 to 14 carbon atoms (e.g., phenoxy), 6) an aromatic heterocycle group (e.g., furyl or thienyl), and the like. The number of the substituents is, for example, 1 to 3.

R²² is preferably 1) an optionally substituted aromatic hydrocarbon group having 6 to 14 carbon atoms (e.g., phenyl, naphthyl and the like) or 2) an optionally substituted 5- or 6-membered aromatic heterocycle group (e.g., furyl, thienyl, pyridyl and the like).

R²² is more preferably 1) an aromatic hydrocarbon group having 6 to 14 carbon atoms (e.g., phenyl, naphthyl and the like) or 2) a 5- or 6-membered aromatic heterocycle group (e.g., furyl, thienyl, pyridyl and the like). Among them, phenyl, furyl, thienyl and the like are preferred.

(2) When X¹ is an oxygen atom, a sulfur atom, —SO—, —SO₂—, —O—SO₂— or —NR³⁶—, wherein the symbol is as defined above;

The hydrocarbon group of the “optionally substituted hydrocarbon group” represented by R²² is preferably an aliphatic hydrocarbon group having 1 to 8 carbon atom(s) (preferably an alkyl group (e.g., methyl, ethyl, propyl or isopropyl), an aromatic aliphatic hydrocarbon group having 7 to 13 carbon atoms (preferably an aralkyl group (e.g., benzyl)) or an aromatic hydrocarbon group having 6 to 14 carbon atoms (preferably phenyl or naphthyl).

The heterocycle group of the “optionally substituted heterocycle group” represented by R²² is preferably a 5- or 6-membered aromatic heterocycle group (e.g., furyl, thienyl, pyridyl and the like).

The substituents for the “optionally substituted hydrocarbon group” and “optionally substituted heterocycle group” are preferably 1) a halogen atom (e.g., fluorine, chlorine, bromine or iodine), 2) an alkyl group having 1 to 4 carbon atom(s) optionally substituted with 1 to 3 halogen atom(s) (e.g., fluorine, chlorine, bromine or iodine) (e.g., methyl, ethyl, trifluoromethyl), 3) an aralkyloxy group having 7 to 10 carbon atoms (e.g., benzyloxy), 4) a 5- or 6-membered aromatic heterocycle group (e.g., pyridyl, oxazolyl, thiazolyl or triazolyl)-alkoxy group having 1 to 6 carbon atom(s) (e.g., methoxy, ethoxy) optionally having 1 or 2 substituent(s) selected from an alkyl having 1 to 3 carbon atom(s) group, a cycloalkyl having 3 to 7 carbon atoms group (e.g., cyclohexyl), furyl, thienyl, phenyl and naphthyl, 5) an aromatic heterocycle group (e.g., furyl, thienyl or pyridyl), 6) an aryloxy group having 6 to 14 carbon atoms (e.g., phenoxy), 7) an alkoxy group having 1 to 4 carbon atom(s) optionally having 1 to 3 halogen atom(s) (e.g., fluorine, chlorine, bromine or iodine) (e.g., methoxy, ethoxy, trifluoromethoxy) and the like. The number of the substituents is, for example, 1 to 3.

R²² is preferably an optionally subsituted aliphatic hydrocarbon group having 1 to 8 carbon atom(s) (preferably an alkyl group (e.g., methyl, ethyl, propyl, isopropyl)), an optionally substituted aromatic aliphatic hydrocarbon group having 7 to 13 carbon atoms (preferably an aralkyl group (e.g., benzyl)) or an optionally substituted heterocycle group (preferably a 5- or 6-membered aromatic heterocycle group (e.g., furyl, thienyl or pyridyl)).

R²² is more preferably an aliphatic hydrocarbon group having 1 to 8 carbon atom(s) (preferably an alkyl group (e.g., methyl, ethyl, propyl or isopropyl)) or an aromatic aliphatic hydrocarbon group having 7 to 13 carbon atoms (preferably an aralkyl group (e.g., benzyl)).

The “bivalent hydrocarbon residue having 1 to 20 carbon atom(s)” represented by W includes “a bivalent non-cyclic hydrocarbon residue”, “a bivalent cyclic hydrocarbon residue”, or a bivalent group obtained by combining one or more “bivalent non-cyclic hydrocarbon residue” with one or more “bivalent cyclic hydrocarbon residue”.

Here, the “bivalent non-cyclic hydrocarbon residue” includes alkylene having 1 to 20 carbon atom(s), alkenylene having 2 to 20 carbon atoms, alkynylene having 2 to 20 carbon atoms and the like.

The “bivalent cyclic hydrocarbon residue” includes a bivalent group obtained by removing any two hydrogen atoms from a cycloalkane having 5 to 20 carbon atoms, a cycloalkene having 5 to 20 carbon atoms or an aromatic hydrocarbon having 6 to 20 carbon atoms (e.g., benzene, naphthalene, indene, anthracene and the like), and the like. The specific examples include 1,2-cyclopentylene, 1,3-cyclopentylene, 1,2-cyclohexylene, 1,3-cyclohexylene, 1,4-cyclohexylene, 1,2-cycloheptylene, 1,3-cycloheptylene, 1,4-cycloheptylene, 3-cyclohexen-1,4-ylene, 3-cyclohexen-1,2-ylene, 2,5-cyclohexadien-1,4-ylene, 1,2-phenylene, 1,3-phenylene, 1,4-phenylene, 1,4-naphthylene, 1,6-naphthylene, 2,6-naphthylene, 2,7-naphthylene, 1,5-indenylene, 2,5-indenylene and the like.

W is preferably “a bivalent hydrocarbon residue having 1 to 20 carbon atom(s)”, more preferably “a bivalent hydrocarbon residue having 1 to 8 carbon atom(s)”. Among them,

(1) C₁₋₈ alkylene (e.g., —CH₂—, —(CH₂)₂—, —(CH₂)₃—, —(CH₂)₄—, —(CH₂),₅—, —(CH₂)₆—, —(CH₂)₇—, —(CH₂)₈—, —CH(CH₃)—, —C(CH₃)₂—, —(CH(CH₃))₂—, —(CH₂)₂C(CH₃)₂—, —(CH₂)₃C(CH₃)₂— and the like;

(2) C₂₋₈ alkenylene (e.g., —CH═CH—, —CH₂—CH═CH—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂— and the like); or

(3) C₂₋₈ alkynylene (e.g., —C≡C—, —CH₂—C≡C—, —CH₂—C≡C—CH₂—CH₂—CH₂— and the like) are preferred.

W is specifically preferably —CH₂—, —(CH₂)₂—, —(CH₂)₃—, —(CH₂)₄—, —CH═CH— or the like.

R ²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring.

The “optionally substituted hydrocarbon group” represented by R²⁸ includes the “optionally substituted hydrocarbon group” exemplified for R²¹.

The “optionally substituted hydrocarbon group” is preferably “an alkyl group having 1 to 4 carbon atom(s)”, “an aryl group having 6 to 10 carbon atoms which optionally has 1 to 3 substituent(s) selected from an alkyl group having 1 to 4 carbon atom(s) and a halogen atom (e.g., fluorine, chlorine, bromine or iodine), or the like. Here, the “alkyl group having 1 to 4 carbon atom(s)” includes methyl, ethyl, propyl, butyl, isobutyl, sec-butyl, t-butyl and the like. Among them, methyl and ethyl are preferred. The “halogen atom” is preferably chlorine. The “aryl group having 6 to 10 carbon atoms” includes phenyl and naphthyl. Among them, phenyl is preferred.

The “optionally substituted hydrocarbon group” and “optionally substituted heterocycle group” represented by R²⁹ and R³⁰ include the “optionally substituted hydrocarbon group” and “optionally substituted heterocycle group” exemplified for R²¹.

The “optionally substituted acyl group” represented by R²⁹ and R³⁰ includes the “optionally substituted acyl group” exemplified as substituents for R²¹.

The ring formed by linking of R²⁹ and R³⁰ includes a 5- to 7-membered cyclic amino group, preferably 1-pyrrolidinyl, 1-piperidinyl, 1-hexamethyleneiminyl, 4-morpholino, 4-thiomorpholino and the like.

R²⁹ and R³⁰ are preferably a hydrogen atom or an alkyl group having 1 to 4 carbon atom(s) (e.g., methyl or ethyl).

R²³ is preferably —OR²⁸ where the symbol is as defined above, and R²⁸ is preferably a hydrogen atom or an alkyl group having 1 to 4 carbon atom(s) (e.g., methyl or ethyl).

Preferable examples of compound (II) include the following compound (A) to (C) and the like.

Compound (A):

a compound wherein

R²¹ is a 5- or 6-membered aromatic heterocycle group (preferably furyl, thienyl, pyridyl, pyrimidinyl, pyrazinyl, oxazolyl, thiazolyl, triazolyl, oxadiazolyl or pyrazolyl) which may be optionally fused with a benzene ring and may optionally have 1 or 2 substituent(s) selected from 1) furyl, thienyl, pyridyl, pyrazinyl, phenyl or naphthyl, each of which optionally has 1 to 3 substituent(s) selected from an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino; and 2) an alkyl group having 1 to 4 carbon atom(s) or a cycloalkyl group having 3 to 7 carbon atoms, each of which optionally has 1 to 3 substituent(s) selected from an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino;

Xa is a bond or —NR²⁶—, wherein R²⁶ is a hydrogen atom or an alkyl group having 1 to 4 carbon atom(s));

ma is 1 or 2;

Ya is an oxygen atom, a sulfur atom, —NH— or —NHCO—;

ring Aa is a benzene ring, a fused aromatic hydrocarbon ring having 9 to 14 carbon atoms (preferably naphthalene and the like) or a 5- or 6-membered aromatic heterocycle (preferably pyridine, isoxazole and the like), each of which may optionally further have 1 to 3 substituent(s) selected from an alkyl group having 1 to 4 carbon atom(s), a hydroxy group, an alkoxy group having 1 to 4 carbon atom(s), an aralkyloxy group having 7 to 10 carbon atoms and a halogen atom;

na is an integer of 1 to 3;

ring B is “a 5-membered aromatic heterocycle containing at least one nitrogen atom as the ring-constituting atoms besides carbon atoms, and optionally further containing one heteroatom selected from an oxygen atom, a sulfur atom and a nitrogen atom” (e.g., pyrrole, pyrazole, imidazole, thiazole, isothiazole, oxazole, isoxazole) which is optionally further substituted with an alkyl group having 1 to 4 carbon atom(s);

X¹ is a bond;

R22 is an aliphatic hydrocarbon group having 1 to 8 carbon atom(s) (preferably an alkyl group), an aromatic hydrocarbon group having 6 to 14 carbon atoms (e.g., phenyl, naphthyl and the like) or 5- or 6-membered aromatic heterocycle group (e.g., furyl, thienyl, pyridyl and the like), each of which may optionally have 1 to 3 substituent(s) selected from 1) a halogen atom (e.g., fluorine, chlorine, bromine, iodine), 2) an alkyl group having 1 to 4 carbon atom(s) optionally substituted with 1 to 3 halogen atom(s) (e.g., fluorine, chlorine, bromine or iodine) (e.g., methyl, trifluoromethyl, propyl or isopropyl), 3) an alkoxy group having 1 to 4 carbon atom(s) optionally substituted with 1 to 3 halogen atom(s) (e.g., fluorine, chlorine, bromine or iodine) (e.g., methoxy or trifluoromethoxy), 4) an aralkyloxy group having 7 to 10 carbon atoms (e.g., benzyloxy), 5) an aryloxy group having 6 to 14 carbon atoms (e.g., phenoxy) and 6) an aromatic heterocycle group (e.g., furyl or thienyl);

W is C₁₋₈ alkylene, C₂₋₈ alkenylene or C₂₋₈ alkynylene; and

R²³ is —OR²⁸, wherein R²⁸ is a hydrogen atom, “an alkyl group having 1 to 4 carbon atom(s)” or “an aryl group having 6 to 10 carbon atoms optionally having 1 to 3 substituent(s) selected from an alkyl group having 1 to 4 carbon atom(s) and a halogen atom (e.g., fluorine, chlorine, bromine or iodine)”, or —NR²⁹R³⁰, wherein R²⁹ and R³⁰ may be the same or different and each is a hydrogen atom or an alkyl group having 1 to 4 carbon atom(s).

Compound (B):

a compound wherein

R21 is a 5- or 6-membered aromatic heterocycle group (preferably furyl, thienyl, pyridyl, pyrimidinyl, pyrazinyl, oxazolyl, thiazolyl, triazolyl, oxadiazolyl or pyrazolyl) which may be optionally fused with a benzene ring and may optionally have 1 or 2 substituent(s) selected from 1) furyl, thienyl, pyridyl, pyrazinyl, phenyl or naphthyl, each of which may optionally have 1 to 3 substituent(s) selected from an alkyl group having 1 to 6 carbon atom(s) optionally substituted with 1 to 3 halogen atom(s) (e.g., fluorine, chlorine, bromine, iodine and the like), an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino; and 2) an alkyl group having 1 to 4 carbon atom(s) or a cycloalkyl group having 3 to 7 carbon atoms, each of which may optionally have 1 to 3 substituent(s) selected from an alkoxy group having 1 to 6 carbon atom(s) optionally substituted with 1 to 3 halogen atom(s) (e.g., fluorine, chlorine, bromine, iodine and the like), a halogen atom (e.g., fluorine, chlorine, bromine, iodine and the like), nitro, hydroxy and amino;

Xa is a bond or —NR²⁶—, wherein R²⁶ is a hydrogen atom or an alkyl group having 1 to 4 carbon atom(s);

ma is 1 or 2;

Ya is an oxygen atom, a sulfur atom, —NH— or —NHCO—;

ring Aa is a benzene ring, a fused aromatic hydrocarbon ring having 9 to 14 carbon atoms (preferably naphthalene and the like) or 5- or 6-membered aromatic heterocycle (preferably pyridine, isoxazole and the like), each of which may optionally have 1 to 3 substituent(s) selected from an alkyl group having 1 to 4 carbon atom(s), a hydroxy group, an alkoxy group having 1 to 4 carbon atom(s), an aralkyloxy group having 7 to 10 carbon atoms and a halogen atom;

na is an integer of 1 to 3;

ring B is “a 5-membered aromatic heterocycle containing at least one nitrogen atom as the ring-constituting atoms besides carbon atoms and optionally containing one heteroatom selected from an oxygen atom, a sulfur atom and a nitrogen atom” (e.g., pyrrole, pyrazole, imidazole, thiazole, isothiazole, oxazole, isoxazole) which may be optionally further substituted with an alkyl group having 1 to 4 carbon atom(s);

X¹ is an oxygen atom;

R²² is an aliphatic hydrocarbon group having 1 to 8 carbon atom(s) (preferably an alkyl group (e.g., methyl, ethyl, propyl, isopropyl)), an aromatic aliphatic hydrocarbon group having 7 to 13 carbon atoms (preferably an aralkyl group (e.g., benzyl)) or a 5- or 6-membered aromatic heterocycle group (e.g., furyl, thienyl, pyridyl and the like), each of which may optionally have 1 to 3 substituent(s) selected from 1) a halogen atom (e.g., fluorine, chlorine, bromine or iodine), 2) an alkyl group having 1 to 4 carbon atom(s) optionally substituted with 1 to 3 halogen atom(s) (e.g., fluorine, chlorine, bromine or iodine) (e.g., methyl, ethyl, trifluoromethyl), 3) an aralkyloxy group having 7 to 10 carbon atoms (e.g, benzyloxy), 4) a 5- or 6-membered aromatic heterocycle (e.g., pyridyl, oxazolyl, thiazolyl or triazolyl)-alkoxy group having 1 to 6 carbon atom(s) (e.g., methoxy, ethoxy) optionally having 1 or 2 substituent(s) selected from an alkyl group having 1 to 3 carbon atom(s), a cycloalkyl group having 3 to 7 carbon atoms (e.g., cyclohexyl), furyl, thienyl, phenyl and naphthyl, 5) an aromatic heterocycle group (e.g., furyl, thienyl, pyridyl), 6) an aryloxy group having 6 to 14 carbon atoms (e.g., phenoxy) and 7) an alkoxy group having 1 to 4 carbon atom(s) optionally substituted with-1 to 3 halogen atom(s) (e.g., fluorine, chlorine, bromine or iodine) (e.g., methoxy, ethoxy or trifluoromethoxy);

W is C₁₋₈ alkylene, C₂₋₈ alkenylene or C₂₋₈ alkynylene; and

R²³ is —OR²⁸, wherein R²⁸ is a hydrogen atom, “an alkyl group having 1 to 4 carbon atom(s)” or “an aryl group having 6 to 10 carbon atoms optionally having 1 to 3 substituent(s) selected from an alkyl group having 1 to 4 carbon atom(s) and a halogen atom (e.g., fluorine, chlorine, bromine or iodine)”, or —NR²⁹R³⁰, wherein R²⁹ and R³⁰ may be the same or different and each is a hydrogen atom or an alkyl group having 1 to 4 carbon atom(s).

Compound(C):

(11) 3-[3-ethoxy-1-[4-(2-phenyl-4-thiazolylmethoxy)benzyl]-1H-pyrazol-4-yl]propionic acid;

(12) 3-[3-ethoxy-1-[4-(2-phenyl-4-oxazolylmethoxy)benzyl]-1H-pyrazol-4-yl]propionic acid;

(13) 3-[3-ethoxy-l-[4-[3-methyl-1-(2-pyridyl)-1H-pyrazol-4-yl]methoxy]benzyl]-1H-pyrazol-4-yl]propionic acid;

(14) 3-[1-[4-(5-methyl-2-phenyl-4-oxazolylmethoxy)benzyl]-3-(2-thienyl)-1H-pyrazol-4-yl]propionic acid;

(15) 3-[1-[4-(2-phenyl-4-thiazolylmethoxy)benzyl]-3-(2-thienyl)-1H-pyrazol-4-yl]propionic acid; or

(16) 3-[1-[4-(2-phenyl-4-oxazolylmethoxy)benzyl]-3-(2-thienyl)-1H-pyrazol-4-yl]propionic acid.

Hereinafter these compounds are sometimes abbreviated as merely compound (11), compound (12) and the like.

A salt of compound (II) includes salts similar to the above-mentioned salts of pioglitazone. Specifically, a sodium salt, a potassium salt, a hydrochloride and the like are preferred.

The compound (II) can be produced, for example, according to the method disclosed in WO01/38325.

In the present invention, although an ABCA1 mRNA expression-promoting agent, an LXRα mRNA expression-promoting agent, an ABCG1 mRNA expression-promoting agent, a cholesterol efflux-promoting agent and a cholesteryl ester accumulation-inhibiting agent, each of which comprises “pioglitazone or a salt thereof” or “a PPARδ modulator”, may be the active ingredient “pioglitazone or a salt thereof” or “PPARδ modulator” itself, they are generally produced by mixing the active ingredient and a pharmacologically acceptable carrier according to a method known per se [e.g. a conventional method in the art of pharmaceutical technique, for example, a method disclosed in The Pharmacopoeia of Japan (e.g., 13th edition) and the like].

Here, the dosage forms of the ABCA1 mRNA expression-promoting agent, LXRα mRNA expression-promoting agent, ABCG1 mRNA expression-promoting agent, cholesterol efflux-promoting agent and cholesteryl ester accumulation inhibiting agent include an oral preparation such as tablets (including sublingual tablets and buccal disintegrating tablets), capsules (including soft capsules and microcapsules), powders, granules, troches, syrups and the like; and a parenteral preparation such as injections (e.g., subcutaneous injections, intravenous injections, intramuscular injections, intraperitoneal injections, drops and the like), an external preparation (e.g., a percutaneous preparation, ointments and the like), suppositories (e.g., rectal suppositories, vaginal suppositories and the like), pellets, a nasal agent, a transpulmonary agent (inhalations), eye drops and the like. These preparations may be a controlled release preparation such as a quick release preparation or a sustained release preparation and the like (e.g., sustained release microcapsules and the like).

Hereinafter production methods for an oral preparation and a parenteral preparation are specifically explained.

The oral preparation is produced by adding, for example, an excipient (e.g., lactose, sucrose, starch, D-mannitol, xylitol, sorbitol, erithritol, crystalline cellulose, light anhydrous silicic acid and the like), a disintegrator (e.g., calcium carbonate, starch, carboxymethyl cellulose, carboxymethyl cellulose calcium, low substituted hydroxypropyl cellulose, croscarmellose sodium, carboxymethyl starch sodium, light anhydrous silicic acid and the like), a binder (e.g., alpha starch, gum arabic, carboxymethyl cellulose, hydroxypropyl cellulose, hydroxypropylmethyl cellulose, polyvinylpyrrolidone, crystalline cellulose, methylcellulose, sucrose, D-mannitol, trehalose, dextrin and the like) or a lubricant (e.g., talc, magnesium stearate, calcium stearate, colloidal silica, polyethyleneglycol 6000 and the like) to an active ingredient and compression molding the mixture. In order to accelerate dissolution of the active ingredient, acids such as hydrochloric acid, phosphoric acid, malonic acid, succinic acid, DL-malic acid, tartaric acid, maleic acid, fumaric acid, citric acid and the like or bases such as sodium carbonate, sodium hydrogencarbonate, sodium citrate, sodium tartrate and the like may be also added to the oral preparation.

Furthermore, for the purpose of masking of taste, enteric property or sustained-release, the oral preparation may be coated according to a method known per se. The coating agent used includes an enteric polymer (e.g., acetate phthalate cellulose, methacrylic acid copolymer L, methacrylic acid copolymer LD, methacrylic acid copolymer S, hydroxypropylmethyl cellulose phthalate, hydroxypropylmethyl cellulose acetate succinate, carboxymethylethyl cellulose and the like), a gastric-soluble polymer (e.g., polyvinyl acetal diethylaminoacetate, aminoalkyl methacrylate copolymer E and the like), a water-soluble polymer (e.g., hydroxypropyl cellulose, hydroxypropylmethyl cellulose and the like), a water-insoluble polymer (e.g., ethyl cellulose, aminoalkyl methacrylate copolymer RS, ethyl acrylate-methyl methacrylate copolymer and the like), a wax and the like. During the coating, a plasticizer such as polyethyleneglycol, an opacifying agent such as titanium oxide, iron sesquioxide and the like may be used together with the coating agent.

The injection is produced by dissolving, suspending or emulsifying the active ingredient in an aqueous solvent (e.g., distilled water, saline, Ringer's solution and the like) or an oily solvent (e.g., a vegetable oil such as olive oil, sesame oil, cottonseed oil, corn oil and the like; propylene glycol, macrogol, tricaprylin and the like) and the like together with a dispersing agent (e.g., Tween 80 (manufactured by Atras Powder, USA), HCO 60 (manufactured by Nikko Chemicals Co., Ltd.), polyethyleneglycol, carboxymethylcellulose, sodium alginate and the like), a preserving agent (e.g., methylparaben, propylparaben, benzyl alcohol, chlorobutanol, phenol and the like), an isotonicity agent (e.g., sodium chloride, glycerine, D-sorbitol, D-mannitol, xylitol, glucose, fructose and the like) and the like.

During the above-mentioned process, if desired, additives such as a dissolving agent (e.g., sodium salicylate, sodium acetate, polyethyleneglycol, propyleneglycol, D-mannitol, trehalose, benzyl benzoate, ethanol, trisaminomethane, cholesterol, triethanolamine, sodium carbonate, sodium citrate and the like), a suspending agent (e.g., a surfactant such as stearyl triethanolamine, sodium laurylsulfate, laurylaminopropionate, lecithin, benzalkonium chloride, benzethonium chloride and glycerine monostearate; a hydrophilic polymer such as polyvinylalcohol, polyvinylpyrrolidone, carboxymethylcellulose sodium, methylcellulose, hydroxymethylcellulose, hydroxyethylcellulose and hydroxypropylcellulose, and the like), a buffer (e.g., a buffer solution such as phosphate, acetate, carbonate and citrate, and the like), a stabilizing agent (e.g., human serum albumin and the like), a soothing agent (e.g., propyleneglycol, lidocaine hydrochloride, benzylalcohol and the like) and an antiseptic (e.g., parahydroxybenzoic acid esters, chlorobutanol, benzalkonium chloride, benzylalcohol, phenethylalcohol, dehydroacetic acid, sorbic acid and the like) may be used.

The external preparation is produced by formulating the active ingredient into solid, semi-solid or liquid composition. For example, the solid composition is produced by pulverizing the active ingredient as it is, or by adding an excipient (e.g., lactose, D-mannitol, starch, crystalline cellulose, sucrose and the like), a thickener (e.g., natural rubbers, cellulose derivatives, acrylic acid polymers and the like) and the like to the active ingredient, mixing them and then pulverizing the mixture. The liquid composition is produced almost similarly to the injection. The semi-solid composition is preferably an aqueous or oily gel, or an ointment form. All of these compositions may also contain a pH modulating agent (e.g., phosphoric acid, citric acid, hydrochloric acid, sodium hydroxide and the like), an antiseptic (e.g., parahydroxybenzoic acid esters, chlorobutanol, benzalkonium chloride, benzylalcohol, phenethylalcohol, dehydroacetic acid, sorbic acid and the like), and the like.

The suppository is produced by formulating the active ingredient into an oily or aqueous solid, semi-solid or liquid composition. Oily bases used in production of the composition include glyceride of higher fatty acid [e.g., cacao butter, Witepsols (manufactured by Hüls Aktiengesellschaft, Germany) and the like], medium fatty acid triglyceride [e.g., Miglyols (manufactured by Hüls Aktiengesellschaft, Germany) and the like], vegetable oil (e.g., sesame oil, soybean oil, cottonseed oil and the like) and the like. Aqueous bases used in production of the composition include polyethyleneglycols, propyleneglycol and the like. Aqueous gel bases used in production of the composition include natural rubbers, cellulose derivatives, vinyl polymers, acrylic acid polymers and the like.

In the ABCA1 mRNA expression-promoting agent, LXRα mRNA expression-promoting agent, ABCG1 mRNA expression-promoting agent, cholesterol efflux-promoting agent and cholesteryl ester accumulation-inhibiting agent, the content of the active ingredient “pioglitazone or a salt thereof” or “PPARδ modulator” is, for example, 0.1 to 100% by weight, preferably 5 to 80% by weight.

The ABCA1 mRNA expression-promoting agent, LXRα mRNA expression-promoting agent, ABCG1 mRNA expression-promoting agent, cholesterol efflux-promoting agent and cholesteryl ester accumulation-inhibiting agent of the present invention have low toxicity, and can be used safely for mammals (e.g., human, mouse, rat, rabbit, dog, cat, cattle, horse, pig, monkey and the like) orally or parenterally.

The dose of the ABCA1 mRNA expression-promoting agent, LXRα mRNA expression-promoting agent, ABCG1 mRNA expression-promoting agent, cholesterol efflux-promoting agent and cholesteryl ester accumulation-inhibiting agent of the present invention may follow the dose of the active ingredient “pioglitazone or a salt thereof” or “PPARδ modulator”, and can be suitably selected depending on the subject to be administered, age and body weight of the subject, condition, administration period, dosage form, administration method and the like.

For example, when the ABCA1 mRNA expression-promoting agent, LXRα mRNA expression-promoting agent, ABCG1 mRNA expression-promoting agent, cholesterol efflux-promoting agent or cholesteryl ester accumulation-inhibiting agent is administered to an adult (body weight 50 kg), the daily dose is generally 0.01 to 1000 mg, preferably 0.1 to 600 mg of the active ingredient “pioglitazone or a salt thereof” or “PPARδ modulator”. Such daily dose may be administered in one to several doses.

Specifically, when the ABCA1 mRNA expression-promoting agent, LXRα mRNA expression-promoting agent, ABCG1 mRNA expression-promoting agent, cholesterol efflux-promoting agent or cholesteryl ester accumulation-inhibiting agent comprising the “pioglitazone or a salt thereof (preferably hydrochloride)” is orally administered to an adult (body weight 50 kg), the daily dose is generally 7.5 to 60 mg, preferably 15 to 45 mg of the “pioglitazone or a salt thereof”. Such daily dose may be administered in one or two doses.

When the ABCA1 mRNA expression-promoting agent, LXRα mRNA expression-promoting agent, ABCG1 mRNA expression-promoting agent, cholesterol efflux-promoting agent or cholesteryl ester accumulation-inhibiting agent comprising the “compound (I) or a salt thereof” as the “PPARδ modulator” is orally administrated to an adult (body weight 50 kg), the daily dose is generally 5 to 500 mg, preferably 10 to 200 mg of the “compound (I) or a salt thereof”. Such daily dose may be administered in one or two doses.

When the ABCA1 mRNA expression-promoting agent, LXRα mRNA expression-promoting agent, ABCG1 mRNA expression-promoting agent, cholesterol efflux-promoting agent or cholesteryl ester accumulation-inhibiting agent comprising the “compound (II) or a salt thereof” as the “PPARδ modulator” is orally administrated to an adult (body weight 50 kg), the daily dose is generally 2 to 500 mg, preferably 5 to 100 mg of the “compound (II) or a salt thereof”. Such daily dose may be administered in one or two doses.

The ABCA1 mRNA expression-promoting agent of the present invention can increase the intracellular content of ABCA1. Thus increased ABCA1 can bind to apoprotein (e.g., apoAI, apoAII and the like) or apolipoprotein (e.g., high density lipoprotein, HDL) present in a living body to carry intracellular cholesterol out of cells. The cholesterol thus carried out of cells is then transported to a tissue having low cholesterol content. Therefore, the ABCA1 mRNA expression-promoting agent of the present invention is useful for control of cholesterol distribution in the body.

Therefore, based on the intracellular cholesterol-efflux effect, the ABCA1 mRNA expression-promoting agent of the present invention is useful as, for example, an agent for prevention or treatment or a development inhibitor (including inhibition of development of arterial sclerosis in type II diabetes mellitus and the like) of diseases such as hypo-HDL cholesterolemia; Tangier disease; coronary artery disease (e.g., myocardial infarction, angina pectoris, asymptomatic myocardial ischemia and the like); carotid arterial sclerosis; cerebrovascular disease (e.g., apoplectic stroke, cerebral infarction and the like); arteriosclerosis obliterans; fatty liver; hepatocirrhosis; diabetic complication; dermatologic disease; xanthomatosis; arthrosis; proliferative disease; peripheral arterial obstruction; ischemic peripheral circulatory failure; obesity; cerebrotendinous xanthomatosis (CTX); glomerular nephritis; vascular thickening;.vascular thickening after intervention (percutaneous coronary plasty, percutaneous coronary revascularization, detention of stent, coronary endoscopy, intravascular sonication, percutaneous transluminal coronary thrombolytic therapy and the like); vascular reocclusion and restenosis after bypass operation; nephropathy, nephritis and pancreatitis, which highly relate to hyperlipidemia; hyperlipidemia (e.g., postprandial hyperlipidemia); intermittent claudication; deep venous thrombosis; malaria encephalitis and the like.

Furthermore, based on the cholesterol transport effect to a tissue having a low cholesterol content, the ABCA1 mRNA expression promoting agent of the present invention is useful as, for example, an agent for prevention or treatment of Alzheimer's disease, wound, diseases accompanied with ateliosis and the like; a healing promoting agent after accident, operation including organ transplantation.

Moreover, the ABCA1 mRNA expression-promoting agent of the present invention is useful as an NO-production-promoting agent, vascular endothelial disease ameliorating agent and the like.

The LXRα mRNA expression-promoting agent of the present invention can increase the intracellular content of LXRα. Since the LXRα allows ABCA1 mRNA to be expressed, the LXRα mRNA expression-promoting agent of the present invention is useful as an agent for prevention or treatment of the various diseases exemplified hereinbefore as diseases for which the ABCA1 mRNA expression-promoting agent of the present invention is useful.

The ABCG1 mRNA expression-promoting agent of the present invention can increase the intracellular content of ABCG1. Thus-increased ABCG1 can bind to apoprotein (e.g., apoAI, apoAII and the like) or apolipoprotein (e.g., high density lipoprotein, HDL) present in a living body to carry intracellular cholesterol out of cells. The cholesterol thus carried out of cells is then transported to a tissue having low cholesterol content. Therefore, the ABCG1 mRNA expression-promoting agent of the present invention is useful as an agent for prevention or treatment of the various diseases exemplified hereinbefore as diseases for which the ABCA1 mRNA expression-promoting agent of the present invention is useful.

The cholesterol efflux-promoting agent and cholesteryl ester accumulation-inhibiting agent of the present invention are useful as an agent for prevention or treatment of the various diseases exemplified hereinbefore as diseases for which the ABCA1 mRNA expression-promoting agent of the present invention is useful.

A “PPARγ modulator” used for the present invention may be any substance capable of activating or suppressing the function of peroxisome proliferator-activated receptor gamma (PPARγ), and specific examples thereof include a PPARγ agonist (including a partial agonist), a PPARγ antagonist (including a partial antagonist) and the like. Furthermore, the “PPARγ modulator” is preferably a substance that shows obvious effect in vitro at a concentration of 10 μM or less.

Preferable examples of the “PPARγ modulator” include the above-mentioned “pioglitazone or a salt thereof”, “compound (I) or a salt thereof” and “compound (II) or a salt thereof“; rosiglitazone;

4-[4-[2-(5-methyl-2-phenyloxazol-4-yl)ethoxy]benzyl]isooxazolidine-3,5-dione (JTT-501);

5-[[3,4-dihydro-2-(phenylmethyl)-2H-1-benzopyran-6-yl]methyl]-2,4-thiazolidinedione (general name: englitazone);

5-[[4-[3-(5-methyl-2-phenyl-4-oxazolyl)-1oxopropyl]phenyl]methyl]-2,4-thiazolidinedione (general name: dalglitazone/CP-86325);

5-[2-(5-methyl-2-phenyl-4-oxazolylmethyl)benzofuran-5-ylmethyl]-2,4-oxazolidinedione (CP-92768);

5-(2-naphthalenylsulfonyl)-2,4-thiazolidinedione (AY-31637);

4-[(2-naphthalenyl)methyl]-3H-1,2,3,5-oxathiadiazol-2-oxide (AY-30711);

5-[[6-(2-fluorobenzyloxy)-2-naphthyl]methyl]-2,4-thiazolidinedione (MCC-555);

[5-[(2,4-dioxothiazolidin-5-yl)methyl]-2-methoxy-N-[[4-(trifluoromethyl)phenyl]methyl]benzamide (AHG-255);

4-[1-(3,5,5,8,8-pentamethyl-5,6,7,8-tetrahydronaphthalene-2-yl)ethenyl]benzoic acid (LGD1069);

6-[1-(3,5,5,8,8-pentamethyl-5,6,7,8-tetrahydronaphthalen-2-yl)cyclopropyl]nicotinic acid (LG100268);

1,4-bis[4-[(3,5-dioxo-1,2,4-oxadiazolidin-2-yl)methyl]phenoxy]-2-butene (YM-440);

(S)-2-ethoxy-3-[4-(2-{4-methanesulfonyloxyphenyl}ethoxy)phenyl]propionic acid (AZ-242);

(−)3-[4-[2-(phenoxazin-10-yl)ethoxy]phenyl]-2-ethoxypropionic acid (NN-622);

bexarotene; GI-262570; DRF-2593; HQL-975; DN-108; CS-011;

dexlipotam; INS-1; AR-H-0329242; CLX-0901; FK-614; KRP-297;

CRE-16336; NN-2344; BM-13-1258; S-15261; KB-R-7785; DRF-2725; GW-2570; GW-2433; MXC-3255; L-746449; L-767827; L-783281; GW-409544; GW-1536; GW-0072; MBX-102; LY-300512;

LY-510929; BMS-298585 and the like.

These compounds may be used as a salt. Such salt includes salts similar to the salts of “pioglitazone” as mentioned above.

The “PPARγ modulator” is preferably “pioglitazone or a salt thereof (preferably hydrochloride)”, “compound (I) or a salt thereof”, “compound (II) or a salt thereof”, “rosiglitazone or a salt thereof (preferably maleate)” or the like. Among them, “pioglitazone or a salt thereof”, “compound (I) or a salt thereof” and “compound (II) or a salt thereof” are preferred.

A “PPARα modulator” used for the present invention may be any substance capable of activating or suppressing the function of peroxisome proliferator-activated receptor alpha (PPARα), and specific examples thereof include a PPARα agonist (including a partial agonist), a PPARα antagonist (including a partial antagonist) and the like. Furthermore, the “PPARα modulator” is preferably a substance that shows obvious effect in vitro at a concentration of 10 μM or less.

The “PPARα modulator” is preferably the above-mentioned “compound (I) or a salt thereof”, Wy-14643, clofibrate, clofibric acid, fenofibrate, fenofibric acid, bezafibrate, GW-2331, GW-9578, palmitic acid, linolenic acid and the like.

In the present invention, the “PPARγ modulator” and “PPARα modulator” may be the same substance.

Although the “ABCG1 mRNA expression-promoting agent comprising a PPARα modulator” of the present invention may be the active ingredient “PPARα modulator” itself, it is generally produced by mixing the active ingredient and a pharmacologically acceptable carrier according to a method known per se [e.g., conventional method in the art of pharmaceutical technique, for example, a method disclosed in The Pharmacopoeia of Japan (e.g., 13th edition) and the like].

The dosage form of the ABCG1 mRNA expression-promoting agent includes dosage forms similar to those of the ABCA1 mRNA expression promoting agent as mentioned above.

In the ABCG1 mRNA expression-promoting agent, the content of the active ingredient “PPARα modulator” is, for example, 0.1 to 100% by weight, preferably 5 to 80% by weight.

The ABCG1 mRNA expression-promoting agent has low toxicity, and can be safely used for mammals (e.g., human, mouse, rat, rabbit, dog, cat, cattle, horse, pig, monkey and the like) orally or parenterally.

The dose of the ABCG1 mRNA expression-promoting agent may follow the dose of the active ingredient “PPARα modulator”, and can be suitably selected depending on the subject to be administered, age and body weight of the subject, condition, administration period, dosage form, administration method and the like.

For example, the ABCG1 mRNA expression promoting agent is orally administered to an adult (body weight 50 kg), the daily dose is generally 0.01 to 1000 mg, preferably 0.1 to 600 mg of the active ingredient “PPARα modulator”. Such daily dose may be administered in one to several doses.

When the ABCG1 mRNA expression-promoting agent comprising the “compound (I) or a salt thereof” as the “PPARα modulator” is administered to an adult (body weight 50 kg), the daily dose is generally 5 to 500 mg, preferably 10 to 200 mg of the “compound (I) or a salt thereof”. Such daily dose may be administered in one or two doses.

The ABCG1 mRNA expression-promoting agent can increase the intracellular content of ABCG1. Thus increased ABCG1 can bind to apoprotein (e.g., apoAI, apoAII and the like) or apolipoprotein (e.g., high density lipoprotein, HDL) present in a living body to carry intracellular cholesterol out of cells. The cholesterol thus carried out of cells is then transported to a tissue having low cholesterol content. Therefore, the ABCG1 mRNA expression-promoting agent of the present invention is useful as an agent for prevention or treatment of the various diseases exemplified hereinbefore as diseases for which the ABCA1 mRNA expression-promoting agent of the present invention is useful.

Since the PPARα modulator can also increase apoprotein (e.g., apoAI, apoAII and the like) and apolipoprotein (e.g., high density lipoprotein, HDL), it has an excellent intracellular cholesterol-efflux effect.

Although the ACAT-1 mRNA expression-inhibiting agent and CEH mRNA expression-promoting agent of the present invention may be the active ingredient “PPARγ modulator” itself, they are generally produced by mixing the active ingredient and a pharmacologically acceptable carrier according to a method known per se [e.g., a conventional method in the art of pharmaceutical technique, for example, a method disclosed in The Pharmacopoeia of Japan (e.g., 13th edition) and the like].

The dosage form of the ACAT-1 mRNA expression-inhibiting agent and CEH mRNA expression-promoting agent includes dosage forms similar to those of the ABCA1 mRNA expression promoting agent and the like as mentioned above.

In the ACAT-1 mRNA expression-inhibiting agent and CEH mRNA expression-promoting agent, the content of the active ingredient “PPARγ modulator” is, for example, 0.1 to 100% by weight, preferably 5 to 80% by weight.

The ACAT-1 mRNA expression-inhibiting agent and CEH mRNA expression-promoting agent have low toxicity, and can be safely used for mammals (e.g., human, mouse, rat, rabbit, dog, cat, cattle, horse, pig, monkey and the like) orally or parenterally.

The dose of the ACAT-1 mRNA expression-inhibiting agent and CEH mRNA expression-promoting agent may follow the dose of the active ingredient “PPARγ modulator”, and can be suitably selected depending on the subject to be administered, age and body weight of the subject, condition, administration period, dosage form, administration method and the like.

For example, the ACAT-1 mRNA expression-inhibiting agent or CEH mRNA expression-promoting agent is administered to an adult (body weight 50 kg), the daily dose is generally 0.01 to 1000 mg, preferably 0.1 to 600 mg of the active ingredient “PPARγ modulator”. Such daily dose may be administered in one to several doses.

Specifically, the ACAT-1 mRNA expression-inhibiting agent or CEH mRNA expression-promoting agent comprising “pioglitazone or a salt thereof (preferably hydrochloride)” as the “PPARγ modulator” is orally administered to an adult (body weight 50 kg), the daily dose is generally 7.5 to 60 mg, preferably 15 to 45 mg of the “pioglitazone or a salt thereof”. Such daily dose may be administered in one or two doses.

When the ACAT-1 mRNA expression-inhibiting agent or CEH mRNA expression-promoting agent” comprising the “compound (I) or a salt thereof” as the “PPARγ modulator” is orally administered to an adult (body weight 50 kg), the daily dose is generally 5 to 500 mg, preferably 10 to 200 mg of the “compound (I) or a salt thereof”. Such daily dose may be administered in one or two doses.

When the ACAT-1 mRNA expression inhibiting agent or CEH mRNA expression-promoting agent comprising the “compound (II) or a salt thereof” as the “PPARγ modulator” is orally administered to an adult (body weight 50 kg), the daily dose is generally 2 to 500 mg, preferably 5 to 100 mg of the “compound (II) or a salt thereof”. Such daily dose may be administered in one or two doses.

The ACAT-1 mRNA expression-inhibiting agent and CEH mRNA expression-promoting agent of the present invention are useful as an agent for prevention or treatment of the various diseases exemplified hereinbefore as diseases for which the ABCA1 mRNA expression-promoting agent is useful.

The ABCA1 mRNA expression-promoting agent, LXRα mRNA expression-promoting agent, ABCG1 mRNA expression-promoting agent, cholesterol efflux-promoting agent, cholesteryl ester accumulation-inhibiting agent, ACAT-1 mRNA expression-inhibiting agent and CEH mRNA expression-promoting agent of the present invention (hereinafter sometimes abbreviated as the agents of the present invention) can be used in combination with concomitant agents that do not adversely affect the agents of the present invention, for the purpose of “enhancing the effect of the agents of the present invention”, “decreasing an amount of the agents of the present invention to be used”, “decreasing side effect of the agents of the present invention” and the like. The concomitant agents may be a low molecular compound, or a high molecular protein, polypeptide, antibodies, vaccines, or the like.

Such concomitant agents include an “agent for treatment of diabetes mellitus”, “agent for treatment of diabetic complication”, “antiobestic agent”, “agent for treatment of hypertension”, “agent for treatment of hyperlipidemia”, “diuretic”, “antithrombotic agent”, “agent for treatment of Alzheimer disease” and the like. Two or more of these concomitant agents may be used in combination.

The above mentioned “agent for treatment of diabetes mellitus” includes the above-mentioned PPARγ modulator, an insulin secretion promoting drug, biguanide, insulin, aglucosidase inhibitor, β3 agonist and the like.

The insulin secretion-promoting drug includes sulfonylurea. Specific examples of sulfonylurea include tolbutamide, chlorpropamide, tolazamide, acetohexamide, glyclopyramide and an ammonium salt thereof, glibenclamide, gliclazide, 1-butyl-3-methanilylurea, carbutamide, glibornuride, glipizide, gliquidone, glisoxepide, glybuthiazole, glybuzole, glyhexamide, glymidine, glypinamide, phenbutamide, tolcyclamide, glimepiride and the like.

Besides the above-mentioned drugs, the insulin secretion-promoting drug includes N-[[4-(1-methylethyl)cyclohexyl]carbonyl]-D-phenylalanine[Nateglinide], (2S)-2-benzyl-3-(cis-hexahydro-2-isoindolinylcarbonyl)propionic acid calcium dihydrate [Mitiglinide], Repaglinide, GLP (Glucagon-like peptide)-1, GLP-1(7-36)-amide, V8-GLP-1 (LY-307161), Exendin-4 (AC-2993), DPP-728-A, V-411, JT-608 and the like.

The biguanide includes phenformin, metformin, buformin and the like.

The insulin includes animal insulin extracted from pancreas of cattle or pig; semi-synthetic human insulin enzymatically synthesized from insulin extracted from pancreas of pig; human insulin genetic technologically synthesized using Escherichia coli or yeast, and the like. As the insulin, insulin zinc containing 0.45 to 0.9 (w/w) % of zinc; protamine insulin zinc produced from zinc chloride, sulfuric acid protamine and insulin, and the like may be used. Furthermore, the insulin may be a fragment or derivative thereof (e.g., INS-1 and the like).

The insulin includes various types such as super immediate-acting type, immediate-acting type, biphasic type, medium type and long-acting type, and can be selected depending on the pathology of a patient.

The α-glucosidase inhibitor includes acarbose, voglibose, miglitol, emiglitate and the like.

The β3 agonist (β3 adrenaline receptor agonist) includes SR-58611-A, SB-226552, AZ40140 and the like.

Besides the above-mentioned drugs, the “agent for treatment of diabetes mellitus” includes ergoset, pramlintide, leptin, BAY-27-9955, T-1095 and the like.

The “agent for treatment of diabetic complication” includes an aldose reductase inhibitor, glycation inhibitor, protein kinase C inhibitor and the like.

The aldose reductase inhibitor includes torlestat; eparlestat; 3,4-dihydro-2,8-diisopropyl-3-thioxo-2H-1,4-benzoxazine-4-acetic acid; imirestat; zenarestat; 6-fluoro-2,3-dihydro-2′,5′-dioxo-spiro[4H-1-benzopyran-4,4′-imidazolidine]-2-carboxamide (SNK-860); zopolrestat; sorbinil; 1-[(3-bromo-2-benzofuranyl)sulfonyl]-2,4-imidazolidinedione (M-16209); CT-112; NZ-314; ARI-509 and the like.

The glycation inhibitor includes pimagedine and the like.

The protein kinase C inhibitor includes NGF, LY-333531 and the like.

Besides the above-mentioned drugs, the “agent for treatment of diabetic complication” includes alprostadil, tiapride hydrochloride, cilostazol, mexiletine hydrochloride, ethyl icosapentate, memantine, pimagedline (ALT-711), neurotrophic factor (e.g., nerve growth factor (NGF), brain-derived nerve growth factor (BDNF), neurotrophic factor 3 (NT-3)), an increasing agent or enhancing agent thereof and the like) and the like.

The “antiobestic agent” includes a lipase inhibitor, anorexic and the like.

The lipase inhibitor includes orlistat and the like.

The anorexic includes dexfenfluramine, fluoxetine, sibutramine, biamine and the like.

The “agent for treatment of hypertension” includes an angiotensin-converting enzyme inhibitor, calcium antagonist, potassium channel opener, angiotensin II antagonist and the like.

The angiotensin-converting enzyme inhibitor includes captopril, enalapril, alacepril, delapril, ramipril, lisinopril, imidapril, benazepril, ceronapril, silazapril, enalaprilate, fosinopril, movertipril, perindopril, quinapril, spirapril, temocapril, trandolapril, manidipine and the like.

The calcium antagonist includes nifedipine, amlodipine, efonidipine, nicardipine and the like.

The potassium channel opener includes levcromakalim, L-27152, AL-0671, NIP-121 and the like.

The angiotensin II antagonist includes losartan, candesartan cilexetil, varsartan, irbesartan, (5-methyl-2-oxo-1,3-dioxolen-4-yl)methyl 4-(1-hydroxy-1-methylethyl)-2-propyl-1-[2′-(1H-tetrazol-5-yl)biphenyl-4-ylmethyl]imidazole-5-carboxylate (CS-866), E4177 and the like.

The “agent for treatment of hyperlipidemia” includes an HMG-CoA reductase inhibitor, fibrate compound, squalene synthase inhibitor, ACAT inhibitor and the like.

The HMG-CoA reductase inhibitor includes pravastatin, simvastatin, lovastatin, atorvastatin, fluvastatin, lipantil, cerivastatin, itavastatin, ZD-4522 or a salt thereof (e.g., sodium salt and the like) and the like.

The fibrate compound includes bezafibrate, beclobrate, binifibrate, ciprofibrate, clinofibrate, clofibrate, clofibric acid, etofibrate, fenofibrate, gemfibrozil, nicofibrate, pirifibrate, ronifibrate, simfibrate, theofibrate and the like.

The squalene synthase inhibitor includes a compound described in WO97/10224 such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like.

The ACAT inhibitor includes avasimibe, eflucimibe and the like.

Beside the above-mentioned drugs, the “agent for treatment of hyperlipidemia” includes anion exchange resin (e.g., cholestyramine and the like), probucol, nicotinic acid agent (e.g., nicomol, niceritrol and the like), ethyl icosapentate, vegetable sterol (e.g., soysterol, γ-oryzanol and the like) and the like.

The “diuretic” includes a xanthine derivative preparation, thiazide preparation, antialdosterone preparation, carbonic anhydrase inhibitor, chlorobenzenesulfonamide preparation and the like.

The xanthine derivative preparation includes theobromine and sodium salicylate, theobromine and calcium salicylate, and the like.

The thiazide preparation includes ethiazide, cyclopenthiazide, trichloromethiazide, hydrochlorothiazide, hydroflumethiazide, benzylhydrochlorothiazide, penflutizide, polythiazide, methyclothiazide and the like.

The antialdosterone preparation includes spironolactone, triamterene and the like.

The carbonic anhydrase inhibitor includes acetazolamide and the like.

The chlorobenzenesulfonamide preparation includes chlorotalidone, mefruside, indapamide and the like.

Besides the above-mentioned drugs, the “diuretic” includes azosemide, isosorbide, ethacrynic acid, piretanide, bumetanide, furosemide and the like.

The “antithrombotic agent” includes heparin, warfarin, antithrombin agent, thrombolytic agent, platelet aggregation suppressing agent (anti-platelet agent) and the like.

The heparin includes heparin sodium, heparin calcium, dalteparin sodium and the like.

The warfarin includes warfarin potassium and the like.

The antithrombin agent includes aragatroban and the like.

The thrombolytic agent includes urokinase, tisokinase, alteplase, nateplase, monteplase, pamiteplase and the like.

The platelet aggregation-suppressing agent includes ticlopidine hydrochloride, cilostazol, ethyl icosapentate, beraprost sodium, sarpogrelate hydrochloride and the like.

The “agent for treatment of Alzheimer disease“includes tacrine, donepezil, rivastigmine, galantamine and the like.

The stages for administration of the agents of the present invention and the concomitant agents are not specifically limited, and both agents may be simultaneously administrated to the subject to be administered, or may be individually administrated at an interval. The dose of the concomitant agents may follow the clinically used dose, and can be suitably selected depending on the subject to be administered, age and body weight of the subject, condition, administration period, dosage form, administration method, combination and the like.

The dosage form of the concomitant agents is not specifically limited as long as the agents of the present invention and the concomitant agents are in combination when they are administered. Such dosage form include 1) administration of a single preparation obtained by formulating the agent of the present invention and the concomitant agent together, 2) simultaneous administration of two kinds of preparations via the same route, wherein the two kinds of preparations are obtained by separately formulating the agent of the present invention and the concomitant agent, 3) administration of two kinds of preparations at an interval via the same route, wherein the two kinds of preparations are obtained by separately formulating the agent of the present invention and the concomitant agent, 4) simultaneous administration of two kinds of preparations via the different routes, wherein the two kinds of preparations are obtained by separately formulating the agent of the present invention and the concomitant agent, 5) administration of two kinds of preparations at an interval via the different routes, wherein the two kinds of preparations are obtained by separately formulating the agent of the present invention and the concomitant agent, (e.g., administration in the order of the agent of the present invention and then the concomitant agent, or in the reverse order) and the like.

The combination ratio of the agent of present invention and the concomitant agent can be suitably selected depending on the subject to be administered, age and body weight of the subject, condition, administration period, dosage form, administration method, and the like.

For example, the concomitant agent may be used in an amount of 0.0001 to 10000 part(s) by weight per 1 part by weight of the agent of the present invention.

The activity of the ABCA1 mRNA expression-promoting agent of the present invention can be confirmed, for example, according to the following method.

Human-derived cells are suspended in a PMA (phorbol 12-myristate 13-acetate) -containing RPMI1640 medium (containing serum), dispensed into plates and cultured until differentiation.

The medium is removed, and a β-VLDL-OA-BSA-containing RPMI1640 medium to which the test compound has been added, is added to the plate and cultured.

Whole RNA is extracted from the cultured cells using RNeasy Mini Kit (QIAGEN). Then, cDNA is synthesized by reverse-transcription reaction of the RNA using TaqMan Reverse Transcription Reagents (Applied Biosystems). Using the obtained reverse-transcription reaction liquid as template DNA and according to TaqMan PCR method using TaqMan Universal PCR Master Mix, expression amounts of ABCA1 mRNA and glyceraldehyde-3-phosphate dehydrogenase (G3PDH) mRNA as an internal standard are measured.

In the above mentioned method, for the measurement of the expression amount of ABCA1 mRNA, oligo DNAs (designed using the Primer Express Program provided by Applied Biosystems) represented by

5′-GCTGAGCTACCCACCCTATGAAC-3′ (SEQ ID NO: 1),

5′-TAATCCCCTGAACCCAAGGAA-3′ (SEQ ID NO: 2) and

5′-TGCCATTTTCCAAATAAAGCCATGCCC-3′ (SEQ ID NO: 3), are used as a sense strand primer, an antisense strand primer and a TaqMan probe, respectively.

For the TaqMan probe used for the measurement of the expression amount of ABCA1 mRNA, 6-carboxyfluorescein (FAM) is used as a reporter dye, and 6-carboxy-N,N,N′,N′-tetramethylrhodamine (TAMRA) is used as a quencher dye. For that purpose, for example, 6FAM is added to the 5′-terminus, and TAMRA is added to the 3′-terminus.

The measurement of the G3PDH mRNA expression is carried out using TaqMan G3PDH Control Reagents (Applied Biosystems), and VIC is used as a reporter dye and TAMRA is used as a quencher dye.

Real-time quantitative PCR reaction is carried out by 40 cycles, wherein one cycle comprises reaction of 50° C. for 2 minutes and at 95° C. for 10 minutes, followed by keeping at 95° C. for 15 seconds and reaction at 60° C. for 1 minute, using ABI PRISM 7700 Sequence Detector (Applied Biosystems).

Based on the ratio of the expression amount of ABCA1 mRNA relative to the expression amount of G3PDH mRNA, ABCA1 mRNA expression promoting activity of test compounds can be confirmed.

The activity of the LXRα mRNA expression-promoting agent of the present invention can be confirmed, for example, according to the following method.

The activity of the LXRα mRNA expression-promoting agent can be confirmed by a method similar to that for the above-mentioned ABCA1 mRNA expression-promoting agent, except for measuring the expression amount of LXRα mRNA by using oligo DNAs represented by

5′-ACACCTACATGCGTCGCAAGT-3′ (SEQ ID NO: 4),

5′-TCTTGCCGCTTCAGTTTCTTC-3′ (SEQ ID. NO: 5) and

5′-TGTGTCCTGTCAGAAGAACAGATCCGCC-3′ (SEQ ID NO: 6)

(designed using the Primer Express Program provided by Applied Biosystems) are used as a sense strand primer, an antisense strand primer and a TaqMan probe, respectively.

The activity of the ABCG1 mRNA expression-promoting agent of the present invention can be confirmed, for example, according to the following method.

The activity of the ABCG1 mRNA expression-promoting agent of the present invention can be confirmed by a similar method to that for the above-mentioned ABCA1 mRNA expression-promoting agent, except for measuring the expression amount of ABCG1 mRNA by using oligo DNAs represented by

5′-TGGAAAATGCCAAGCTGTACCT-3′ (SEQ ID NO: 7),

5′-CGGATTTTGTACCTGAGGAC-3′ (SEQ ID NO: 8) and

5′-TTCATCTCCCTCCGCCTCATTGCCTA-3′ (SEQ ID NO: 9) (designed using the Primer Express Program provided by Applied Biosystems) are used as a sense strand primer, an antisense strand primer and a TaqMan probe, respectively.

The activity of the cholesterol efflux-promoting agent of the present invention can be confirmed, for example, according to the following method.

Human-derived cells are suspended into a PMA (phorbol 12-myristate 13-acetate)-containing RPMI1640 medium (containing serum), dispensed in plates and cultured until differentiation.

The medium is removed, and [³H]cholesteryl linolate-β-VLDL-OA-BSA-containing RPMI1640 medium, to which a test compound has been added, is added to the plate and cultured.

The plate is washed with a buffer solution, and then a RPMI1640 medium containing a test compound and apoA1 is added thereto and cultured.

Thus-obtained culture solution is centrifuged. The radioactivity of the supernatant is measured using a liquid scintillation counter to confirm the cholesterol efflux-promoting activity of the test compound.

### BEST MODE FOR CARRYING OUT THE INVENTION

Hereinafter the present invention is more specifically explained with referring to Examples and Experimental Examples which do not limit the present invention.

The SEQ ID NOs in the Sequence Listing of the present description represent the following sequences.

[SEQ ID NO: 1] shows a sense strand primer, which is used to amplify cDNA encoding human ABCA1.

[SEQ ID NO: 2] shows an antisense strand primer, which is used to amplify cDNA encoding human ABCA1.

[SEQ ID NO: 3] shows a TaqMan probe, which is used to quantify cDNA encoding human ABCA1.

[SEQ ID NO: 4] shows a sense strand primer, which is used to amplify cDNA encoding human LXRα.

[SEQ ID NO: 5] shows an antisense strand primer, which is used to amplify cDNA encoding human LXRα.

[SEQ ID NO: 6] shows a TaqMan probe, which is used to quantify cDNA encoding the amplified human LXRα.

[SEQ ID NO: 7] shows a sense strand primer, which is used to amplify cDNA encoding human ABCG1.

[SEQ ID NO: 8] shows an antisense strand primer, which is used to amplify cDNA encoding human ABCG1.

[SEQ ID NO: 9] shows a TaqMan probe, which is used to quantify cDNA encoding the amplified human ABCG1.

[SEQ ID NO: 10] shows a sense strand primer, which is used to amplify cDNA encoding mouse LXRα.

[SEQ ID NO: 11] shows an antisense strand primer, which is used to amplify cDNA encoding mouse LXRα.

[SEQ ID NO: 12] shows a TaqMan probe, which is used to quantify cDNA encoding the amplified mouse LXRα.

[SEQ ID NO: 13] shows a sense strand primer, which is used to amplify cDNA encoding mouse ABCG1.

[SEQ ID NO: 14] shows an antisense strand primer, which is used to amplify cDNA encoding mouse ABCG1.

[SEQ ID NO: 15] shows a TaqMan probe, which is used to quantify cDNA encoding the amplified mouse ABCG1.

[SEQ ID NO: 16] shows a sense strand primer, which is used to amplify cDNA encoding mouse acidic ribosomal protein (36B4).

[SEQ ID NO: 17] shows an antisense strand primer, which is used to amplify cDNA encoding mouse acidic ribosomal protein (36B4).

[SEQ ID NO: 18] shows a TaqMan probe, which is used to quantify cDNA encoding the amplified mouse acidic ribosomal protein (36B4).

[SEQ ID NO: 19] shows a sense strand primer, which is used to amplify cDNA encoding human ACAT-1.

[SEQ ID NO: 20] shows an antisense strand primer, which is used to amplify cDNA encoding human ACAT-1.

[SEQ ID NO: 21] shows a TaqMan probe, which is used to quantify cDNA encoding the amplified human ACAT-1.

[SEQ ID NO: 22] shows a sense strand primer, which is used to amplify cDNA encoding human CEH.

[SEQ ID NO: 23] shows an antisense strand primer, which is used to amplify cDNA encoding human CEH.

[SEQ ID NO: 24] shows a TaqMan probe, which is used to quantify cDNA encoding the amplified human CEH.

### EXAMPLES

### Example 1

### Production of Tablets

Pioglitazone hydrochloride (2479.5 g: 2250 g as pioglitazone), lactose (13930.5 g) and carboxymethylcellulose calcium (carmelose calcium) (540 g) were fed into a fluidized-bed granulation and drying machine (manufactured by Powerex Corporation), mixed with preheating, and sprayed with an aqueous solution (7500 g) in which hydroxypropylcellulose (450 g) had been dissolved, to give granulated powder. The obtained granulated powder (16820 g) was passed through a cutter mill (manufactured by Showa Kagaku Kikai Kosakusho) to give sized powder. The obtained sized powder (16530 g), carmelose calcium (513 g) and magnesium stearate (57 g) were formed into mixed powder using a tumbler mixer (manufactured by Showa Kagaku Kikai Kosakusho), and then the mixed powder (16800 g) was compressed into tablets by a tableting machine (manufactured by Kikusui Seisakusho) to give 140,000 tablets containing 15 mg of pioglitazone per a tablet and having the following composition.

Composition of a tablet (unit: mg):

| | |
|---|---|
| 1) pioglitazone hydrochloride | 16.53 |
| 2) lactose | 92.87 |
| 3) carmelose calcium | 7.2 |
| 4) hydroxypropylcellulose | 3.0 |
| 5) magnesium stearate | 0.4 |
| Total | 120.0 |

### Example 2

### Production of Tablets

According to the similar method to Example 1, 140,000 tablets containing 30 mg of pioglitazone per a tablet and having the following composition were obtained.

Composition of a tablet (unit: mg):

| | |
|---|---|
| 1) pioglitazone hydrochloride | 33.06 |
| 2) lactose | 76.34 |
| 3) carmelose calcium | 7.2 |
| 4) hydroxypropylcellulose | 3.0 |
| 5) magnesium stearate | 0.4 |
| Total | 120.0 |

### Example 3

### Production of Tablets

According to the similar method to Example 2, 140,000 tablets containing 45 mg of pioglitazone per a tablet and having the following composition were obtained.

Composition of a tablet (unit: mg):

| | |
|---|---|
| 1) pioglitazone hydrochloride | 49.59 |
| 2) lactose | 114.51 |
| 3) carmelose calcium | 10.8 |
| 4) hydroxypropylcellulose | 4.5 |
| 5) magnesium stearate | 0.6 |
| Total | 180.0 |

### Example 4

### Production of Film Tablets

### Production of Coating Agents

To purified water (4859.1 g) were dissolved hydroxypropylmethyl cellulose 2910(TC-5) (403.4 g) and polyethyleneglycol 6000 (81.0 g). Into the obtained solution were dispersed titanium oxide (54.0 g) and yellow iron sesquioxide (1.620 g) to give a coating agent.

### Production of Bare Tablets

Compound (7) (576.0 g), lactose (2513 g), corn starch (356.4 g) and croscarmellose sodium (217.8 g) were fed into a fluidized-bed granulation and drying machine (manufactured by Powerex Corporation), mixed with preheating, and sprayed with an aqueous solution (1963 g) in which hydroxypropylcellulose (138.6 g) had been dissolved, to give granulated powder. The obtained granulated powder (3590 g) was passed through a power mill (manufactured by Showa Kagaku Kikai Kosakusho) to give sized powder. The obtained sized powder (3432 g), corn starch (125.1 g) and magnesium stearate (17.88 g) were mixed in a tumbler mixer (manufactured by Showa Kagaku Kikai Kosakusho), and then the obtained mixed powder (3410 g) was compressed into tablets by a tableting machine (manufactured by Kikusui Seisakusho) to give bare tablets.

### Production of Film Coating Tablets

The obtained bare tablets (27000 tablets) was sprayed with the above-mentioned coating agent in a film coating machine (manufactured by Powerex Corporation) to give 27,000 tablets of film coating tablets containing 16.0 mg of the compound (7) per a tablet and having the following formulation.

Formulation of a tablet (composition per a tablet):
(Bare tablet)

| | | |
|---|---|---|
| 1) compound (7) | 16.0 | mg |
| 2) lactose | 69.8 | mg |
| 3) corn starch | 13.75 | mg |
| 4) croscarmellose sodium | 6.05 | mg |
| 5) hydroxypropylcellulose | 3.85 | mg |
| 6) magnesium stearate | 0.55 | mg |
| Total | 110.0 | mg |

(Film component)

| | | |
|---|---|---|
| 7) hydroxypropylmethyl cellulose 2910 | 2.988 | mg |
| 8) polyethyleneglycol 6000 | 0.6 | mg |
| 9) titanium oxide | 0.4 | mg |
| 10) yellow iron sesquioxide | 0.012 | mg |
| Total | 114.0 | mg |

### Example 5

### Production of Film Tablets

### Production of Bare Tablets

According to the similar method to Example 4 except that the amount to be used of the compound (7) and lactose were 144.0 g and 2945 g respectively, 27,000 tablets of film coating tablets containing 4.0 mg of the compound (7) per a tablet and having the following formulation were obtained.

Formulation of a tablet (Composition per a tablet):
(Bare tablet)

| | | |
|---|---|---|
| 1) compound (7) | 4.0 | mg |
| 2) lactose | 81.8 | mg |
| 3) corn starch | 13.75 | mg |
| 4) croscarmellose sodium | 6.05 | mg |
| 5) hydroxypropylcellulose | 3.85 | mg |
| 6) magnesium stearate | 0.55 | mg |
| Total | 110.0 | mg |

(Film component)

| | | |
|---|---|---|
| 7) hydroxypropylmethyl cellulose 2910 | 2.988 | mg |
| 8) polyethyleneglycol 6000 | 0.6 | mg |
| 9) titanium oxide | 0.4 | mg |
| 10) yellow iron sesquioxide | 0.012 | mg |
| Total | 114.0 | mg |

### Example 6

### Production of Film Tablets

### Production of Bare Tablets

According to the similar method to Example 4 except that the amount to be used of the compound (7) and lactose were 36.0 g and 3053 g respectively, 27,000 tablets of film coating tablets containing 1.0 mg of the compound (7) per a tablet and having the following formulation were obtained.

Formulation of a tablet (Composition per a tablet):
(Bare tablet)

| | | |
|---|---|---|
| 1) compound (7) | 1.0 | mg |
| 2) lactose | 84.8 | mg |
| 3) corn starch | 13.75 | mg |
| 4) croscarmellose sodium | 6.05 | mg |
| 5) hydroxypropylcellulose | 3.85 | mg |
| 6) magnesium stearate | 0.55 | mg |
| Total | 110.0 | mg |

(Film component)

| | | |
|---|---|---|
| 7) hydroxypropylmethyl cellulose 2910 | 2.988 | mg |
| 8) polyethyleneglycol 6000 | 0.6 | mg |
| 9) titanium oxide | 0.4 | mg |
| 10) yellow iron sesquioxide | 0.012 | mg |
| Total | 114.0 | mg |

### Experimental Example 1

### Expression Modulatory Activities for LXRα mRNA, ABCA1 mRNA and ABCG1 mRNA in Human Monocyte-Derived Cells

To a PMA (phorbol 12-myristate 13-acetate, 400 ng/mL)-FBS (fetal bovine serum, 10%)-containing RPMI medium was suspended human monocyte -derived THP-1 cells (ATCC) (7×10⁵ cell/mL), and the obtained suspension was dispensed into a 24-well plate (0.5 mL/well) and cultured for 3 days. After removing the medium, a FBS (fetal bovine serum, 10%)—containing RPMI1640 medium into which a test compound (1 μM) had been added was added to the 24-well plate and the plate was cultured for 24 hours. Whole RNA was extracted from the cultured cells using RNeasy Mini Kit (QIAGEN), and then the expression amounts of LXRα mRNA, ABCA1 mRNA, ABCG1 mRNA and Human acidic ribosomal protein (huPO; 36B4) mRNA as an internal standard were measured by real-time quantitative RT-PCR according to TaqMan PCR method using a TaqMan EZ RT-PCR kit (Applied Biosystems).

In the above-mentioned method, for the measurement of the expression amount of LXRα mRNA, the above-mentioned oligo DNAs represented by SEQ ID NO: 4, SEQ ID NO: 5 and SEQ ID NO: 6 (designed using the Primer Express Program provided by Applied Biosystems) were used as a sense strand primer, an antisense strand primer and a TaqMan probe, respectively.

Furthermore, for the measurement of the expression amount of ABCA1 mRNA, the above-mentioned oligo DNAs represented by SEQ ID NO: 1, SEQ ID NO: 2 and SEQ ID NO: 3 (designed using the Primer Express Program provided by Applied Biosystems) were used as a sense strand primer, an antisense strand primer and a TaqMan probe, respectively.

Moreover, for the measurement of the expression amount of ABCG1 mRNA, the above-mentioned oligo DNAs represented by SEQ ID NO: 7, SEQ ID NO: 8 and SEQ ID NO: 9 (designed using the Primer Express Program provided by Applied Biosystems) were used as a sense strand primer, an antisense strand primer and a TaqMan probe, respectively.

For the TaqMan probes used for the above-mentioned various measurements, 6-carboxyfluorescein (6FAM) as a reporter dye was added to the 5′-terminus, and 6-carboxy-N,N,N′,N′-tetramethylrhodamine (TAMRA) as a quencher dye was added to the 3′-terminus.

The expression amount of Human acidic ribosomal protein (huPO; 36B4) mRNA was measured using Pre-Developed TaqMan Assay Reagents Endogenous Control (Human Acidic ribosomal protein (huPO)) (Applied Biosystems), and VIC was used as a reporter dye and TAMRA was used as a quencher dye.

Real-time quantitative RT-PCR is carried out by 40 cycles, wherein one cycle comprises reaction of 50° C. for 2 minutes and at 60° C. for 30 minutes, keeping at 95° C. for 5 minutes and then at 94° C. for 20 seconds, and reaction at 60° C. for 1 minute, using ABI PRISM 7700 Sequence Detector (Applied Biosystems).

Based on the ratio of the expression amount of the LXRα mRNA, ABCA1 mRNA and ABCG1 mRNA relative to the internal standard (expression amount of Human acidic ribosomal protein mRNA), the expression modulatory activities for the LXRα mRNA, ABCA1 mRNA and ABCG1 mRNA of the test compound were evaluated. The results are shown in Table 1.

TABLE 1

| | Expression amount of mRNA | | |
|---|---|---|---|
| | (Relative ratio % to internal standard) | | |
| Test compound | LXRα | ABCA1 | ABCG1 |
| Not added | 100.0 ± 7.1 | 100.0 ± 7.1 | 100.0 ± 11.7 |
| Pioglitazone | 165.7 ± 4.6 | 108.2 ± 3.5 | 252.0 ± 9.2 |
| hydrochloride | | | |
| Compound (7) | 160.1 ± 9.0 | 113.5 ± 5.5 | 265.5 ± 22.0 |
| Mean ± SEM (n = 3) | | | |

As shown in Table 1, pioglitazone hydrochloride and the compound (7) increased the expression amounts of LXRα mRNA, ABCA1 mRNA and ABCG1 mRNA.

### Experimental Example 2

### Expression Modulatory Activities for LXRα mRNA and ABCG1 mRNA in Mouse Macrophages

A C57BL6/J mouse (male, 10 weeks old) was intraperitoneally administered with 1 mL of a 5% thioglycolic acid medium (Wako Pure Chemical Industries, Ltd.). After 4 days, intraperitoneal cells were collected with phosphate-buffered saline (PBS), and macrophages were separated in a 3% BSA-containing DMEM medium as cells adhesive to a plate. After incubation of the macrophages overnight in a DMEM medium containing 10% human lipoprotein-removed serum, the medium was replaced with a DMEM medium containing a test compound (1 μM) and 10% fetal bovine serum and cultured for 24 hours.

After the medium was removed, whole RNA was extracted from the obtained macrophage using RNeasy Mini Kit (QIAGEN) and then the expression amounts of the LXRαmRNA, ABCG1mRNA and mouse acidic ribosomal protein (36B4) mRNA as an internal standard were measured by real-time quantitative RT-PCR according to TaqMan PCR method using a TaqMan EZ RT-PCR kit (Applied Biosystems).

In the above mentioned method, for the measurement of the expression amount of the LXRα mRNA, oligo DNAs represented by

5′-AGCAACAGTGTAACAGGCGCT-3′ (SEQ ID NO: 10),

5′-ACGATGGCCAGCTCAGTAAAG-3′ (SEQ ID NO: 11) and

5′-CTCAGACCGCCTGCGCGTCA-3′ (SEQ ID NO: 12)

(designed using the Primer Express Program provided by Applied Biosystems) were used as a sense strand primer, an antisense strand primer and a TaqMan probe, respectively.

Furthermore, for the measurement of the expression amount of the ABCG1 mRNA, oligo DNAs represented by

5′-TCAGGAGGCCATGATGGTG-3′ (SEQ ID NO: 13),

5′-CCCGTCTGCCTTCATCCTT-3′ (SEQ ID NO: 14) and

5′-CCGCGCATCTGAAGCTGCAGG-3′ (SEQ ID NO: 15)

(designed using the Primer Express Program provided by Applied Biosystems) were used as a sense strand primer, an antisense strand primer and a TaqMan probe, respectively.

For the TaqMan probes used for the above-mentioned various measurements, 6-carboxyfluorescein (6FAM) as a reporter dye was added to the 5′-terminus, and 6-carboxy-N,N,N′,N′-tetramethylrhodamine (TAMRA) as a quencher dye was added to the 3′-terminus.

For the measurement of the expression amount of mouse acidic ribosomal protein (36B4) mRNA, oligo DNAs represented by

5′-AGACCTCCTTCTTCCAGGCTTT-3′ (SEQ ID NO: 16),

5′-GCTGCACATCACTCAGAATTTCA-3′ (SEQ ID NO: 17) and

5′-TCACCACGAAAATCTCCAGAGGCACC-3′ (SEQ ID NO: 18)

(designed using the Primer Express Program provided by Applied Biosystems) were used as a sense strand primer, an antisense strand primer and a TaqMan probe, respectively. In the TaqMan probes, VIC as a reporter dye was added to the 5′-terminus and TAMRA as a quencher dye was added to the 3′-terminus.

Real-time quantitative PCR was carried out according to the similar manner to Experimental Example 1.

Based on the ratio of the expression amount of the LXRαmRNA and ABCG1mRNA relative to the internal standard (expression amount of mouse acidic ribosomal protein mRNA), the expression modulatory activities for the LXRα mRNA and ABCG1 mRNA of the test compound were evaluated. The results are shown in Table 2.

TABLE 2

Expression amount of mRNA
(Relative ratio % to internal
standard)

| Test compound | LXRα | ABCG1 |
|---|---|---|
| Not added | 100.0 ± 13.6 | 100.0 ± 4.2 |
| Pioglitazone hydrochloride | 145.3 ± 17.9 | 195.2 ± 23.1 |
| Compound (7) | 173.0 ± 9.9 | 159.1 ± 6.1 |
| Mean ± SEM (n = 3) | | |

As shown in Table 2, pioglitazone hydrochloride and the compound (7) increased the expression amounts of LXRα mRNA and ABCG1 mRNA.

### Experimental Example 3

### Foaming-Suppressant Activity in Human Monocyte-Derived Cells

In a RPMI1640 medium containing a test compound (1 μM), PMA (phorbol 12-myristate 13-acetate, 400 ng/ml) and 10% fetal bovine serum was suspended human monocyte-derived THP-1 cells (purchased from Dainippon Pharmaceutical Co., Ltd.). The obtained suspension was dispensed into a 12-well plate (7.2×10⁵ cells) and was cultured for 3 days. The obtained cell was further cultured in a RPMI1640 medium containing the test compound and 0.2 mM oleic acid-0.2% bovine serum albumin complex and rabbit β-VLDL (200 μg cholesterol/mL) for 2 days, and then the total lipids in the cells were extracted with a mixed solution of n-hexane/isopropanol (3:2). The amounts of the total cholesterol (TC) and free cholesterol (FC) in the extract were measured with a enzyme method [using Cholesterol E-Test Wako (trade name, Wako Pure Chemical Industries, Ltd.)], and the amount of cholesteryl ester (CE) was calculated by the difference between the two cholesterol amounts. The intracellular lipid content was then evaluated by dividing the amounts of these lipids by the amount of cell protein (measured using BCA assay kit (Pierce)). The results are shown in Table 3. In the Table, TC represents total cholesterol, FC represents free cholesterol and CE represents cholesteryl ester.

TABLE 3

| | Intracellular lipid content | | |
|---|---|---|---|
| | (μg/mg cell protein) | | |
| Test compound | TC | FC | CE |
| Not added | 114.8 ± 1.8 | 80.8 ± 1.2 | 34.0 ± 2.3 |
| Pioglitazone | 100.3 ± 0.3 | 72.7 ± 0.4 | 27.6 ± 0.5 |
| hydrochloride | | | |
| Compound (7) | 95.8 ± 1.4 | 72.0 ± 1.3 | 23.7 ± 1.9 |
| Mean ± SEM (n = 3) | | | |

As shown in Table 3, pioglitazone hydrochloride and the compound (7) decreased the amounts of total cholesterol, free cholesterol and cholesteryl ester in cells. Based on the decrease in the amount of cholesteryl ester, it was revealed that pioglitazone hydrochloride and the compound (7) have foaming-suppressant activity.

### Experimental Example 4

### Expression Modulatory Activities for ACAT-1 mRNA and CEH mRNA in Human Moncyte-Derived Cells

In a RPMI medium containing a test compound (1 μM), PMA (phorbol 12-myristate 13-acetate, 400 ng/mL)-FBS (fetal bovine serum, 10%) was suspended human monocyte-derived THP-1 cells (purchased from Dainippon Pharmaceutical Co., Ltd.) (7.2×10⁵ cell/mL). The obtained suspension was dispensed into a 24 well plate (0.5 mL/well) and cultured for 2 days (3.6×10⁵ cells). After the medium was removed, a RPMI medium containing the test compound (1 μM), PMA(400 ng/mL)-FBS(10%) was added to the plate and it was cultured further for 1 day.

After the medium was removed, whole RNA was extracted from the obtained cells using RN easy Mini Kit (QIAGEN), and the expression amounts of ACAT-1 mRNA, CEH mRNA and human acidic ribosomal protein (huPO; 36B4) mRNA as an internal standard were measured by real-time quantitative RT-PCR according to TaqMan PCR method using a TaqMan EZ RT-PCR kit (Applied Biosystems).

In the above mentioned method, for the measurement of the expression amount of the ACAT-1mRNA, oligo DNAs represented by

5′-GCAGCTGCTCTGGTTCTCATG-3′ (SEQ ID NO: 19),

5′-TTCTTGCCAGTGGTGTAACATTG-3′ (SEQ ID NO: 20) and

5′-CGGCAGATGCAGCGAAGAGGC-3′ (SEQ ID NO: 21) (designed using the Primer Express Program provided by Applied Biosystems) were used as a sense strand primer, an antisense strand primer and a TaqMan probe, respectively.

Furthermore, for the measurement of the expression amount of the CEH mRNA, oligo DNAs represented by

5′-AGCCCTTGGCTGAGCAAA-3′ (SEQ ID NO: 22),

5′-GTCGCAGGCAGTGAACCAT-3′ (SEQ ID NO: 23) and

5′-TGCTATCACTGCTGGGTGCAAAACCA-3′ (SEQ ID NO: 24)

(designed using the Primer Express Program provided by Applied Biosystems) were used as a sense strand primer, an antisense strand primer and a TaqMan probe, respectively.

For the TaqMan probes used for the above-mentioned various measurements, 6-carboxyfluorescein (6FAM) as a reporter dye was added to the 5′-terminus and 6-carboxy-N,N,N′,N′-tetramethylrhodamine (TAMRA) as a quencher dye was added to the 3′-terminus.

The expression amount of human acidic ribosomal protein (huPO; 36B4) mRNA was measured according to the method similar to Experimental Example 1. Real-time quantitative RT-PCR was carried out similarly to Experimental Example 1.

Based on the ratio of the expression amounts of the ACAT-1 mRNA and CEH mRNA relative to the internal standard (expression amount of human acidic ribosomal protein mRNA), the expression modulatory activities for the ACAT-1 mRNA and CEH mRNA of the test compound were evaluated. The results are shown in Table 4.

TABLE 4

Expression amount of mRNA
(Relative ratio % to internal
standard)

| Test compound | ACAT-1 | CEH |
|---|---|---|
| Not added | 100.0 ± 8.6 | 100.0 ± 13.0 |
| Pioglitazone hydrochloride | 65.8 ± 1.9 | 148.6 ± 9.2 |
| Compound (7) | 66.1 ± 1.5 | 159.4 ± 1.9 |
| Mean ± SEM (n = 3) | | |

As shown in Table 4, pioglitazone hydrochloride and the compound (7) decreased the expression amount of ACAT-1 mRNA and increased the expression amount of CEH mRNA.

### Industrial Applicability

The ABCA1 mRNA expression promoting agent, LXRα mRNA expression promoting agent, ABCG1 mRNA expression-promoting agent, cholesterol efflux-promoting agent, cholesteryl ester accumulation-inhibiting agent, ACAT-1 mRNA expression-inhibiting agent and CEH mRNA expression-promoting agent of the present invention are excellent in the ability to control cholesterol distribution in the body and have low toxicity.

1. An ABCA1 mRNA expression-promoting agent comprising pioglitazone or a salt thereof.

2. An ABCA1 mRNA expression-promoting agent comprising PPARδ modulator.

3. The agent according to claim 2, wherein the PPARδ modulator is a compound represented by the formula: 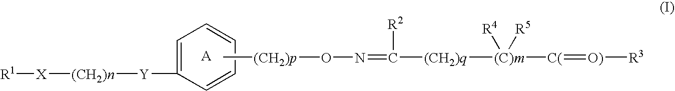
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³is a hydroxy group, —OR⁸, wherein R⁸ is an optionally substituted hydrocarbon group, or —NR⁹R¹⁰ , wherein R9 and R¹⁰ may be the same or different and each is a hydrogen atom, an optionally substituted hydrocarbon group, an optionally substituted heterocycle group or an acyl group or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

4. The agent according to claim 3, wherein the PPARδ modulator is E-4-[4-(5-methyl-2-phenyl-4-oxazolylmethoxy)benzyloxyimino]-4-phenylbutyric acid or a salt thereof.

5. The agent according to claim 2, wherein the PPARδ modulator is a compound represented by the formula: 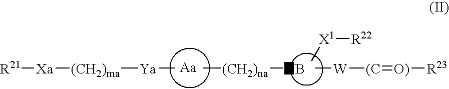
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO₂—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO2—, —O—SO2— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

6. The agent according to claim 5, wherein the PPARδ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

7. An LXRαmRNA expression-promoting agent comprising pioglitazone or a salt thereof.

8. An LXRαmRNA expression-promoting agent comprising PPARδ modulator.

9. The agent according to claim 8, wherein the PPARδ modulator is a compound represented by the formula: 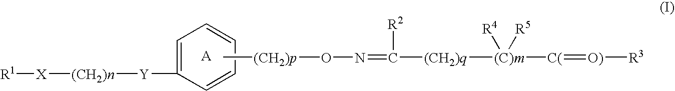
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³ is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

10. The agent according to claim 9, wherein the PPARδ modulator is E-4-[4-(5-methyl-2-phenyl-4-oxazolylmethoxy)benzyloxyimino]-4-phenylbutyric acid or a salt thereof.

11. The agent according to claim 8, wherein the PPARδ modulator is a compound represented by the formula: 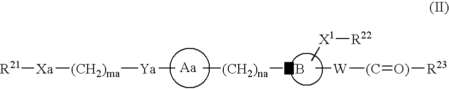
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO₂—, —NR²⁷—, —CONR²⁷— or —NR²⁷CO—, wherein R27 is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

12. The agent according to claim 11, wherein the PPARδ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

13. An ABCG1 mRNA expression-promoting agent comprising pioglitazone or a salt thereof.

14. An ABCG1 mRNA expression-promoting agent comprising PPARδ modulator.

15. The agent according to claim 14, wherein the PPARδ modulator is a compound represented by the formula: 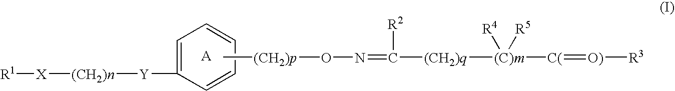
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³is a hydroxy group, —OR⁸ , wherein R⁸ is an optionally substituted hydrocarbon group, or —NR⁹R¹⁰ , wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

16. The agent according to claim 15, wherein the PPARδ modulator is E-4-[4-(5-methyl-2-phenyl-4-oxazolylmethoxy)benzyloxyimino]-4-phenylbutyric acid or a salt thereof.

17. The agent according to claim 14, wherein the PPARδ modulator is a compound represented by the formula: 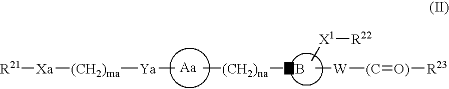
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, SO₂—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

18. The agent according to claim 17, wherein the PPARδ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid; 3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

19. An ABCG1 mRNA expression-promoting agent comprising a PPARα modulator.

20. A cholesterol efflux-promoting agent comprising pioglitazone or a salt thereof.

21. A cholesterol efflux-promoting agent comprising a PPARδ modulator.

22. The agent according to claim 21, wherein the PPARδ modulator is a compound represented by the formula: 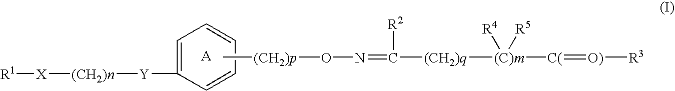
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷ —, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³ is a hydroxy group, —OR⁸ wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

23. The agent according to claim 22, wherein the PPARδ modulator is E-4-[4-(5-methyl-2-phenyl-4-oxazolylmethoxy)benzyloxyimino]-4-phenylbutyric acid or a salt thereof.

24. The agent according to claim 21, wherein the PPARδ modulator is a compound represented by the formula: 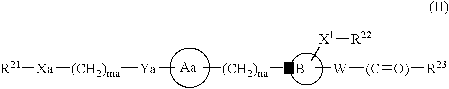
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR 26—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO2—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R22 is a hydrogen atom, an optionally substituted hydrocarbon group or an optionally substituted heterocycle group;
W is a bond or a bivalent hydrocarbon residue having 1 to 20 carbon atom(s);
R²³ is —OR^{28,} wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

25. The agent according to claim 24, wherein the PPARδ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

26. A cholesteryl ester accumulation-inhibiting agent comprising pioglitazone or a salt thereof.

27. A cholesteryl ester accumulation-inhibiting agent comprising PPARδ modulator.

28. The agent according to claim 27, wherein the PPARδ modulator is a compound represented by the formula: 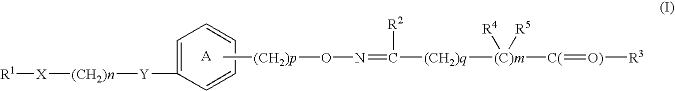
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³ is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

29. The agent according to claim 28, wherein the PPARδ modulator is E-4-[4-(5-methyl-2-phenyl-4-oxazolylmethoxy)benzyloxyimino]-4-phenylbutyric acid or a salt thereof.

30. The agent according to claim 27, wherein the PPARδ modulator is a compound represented by the formula: 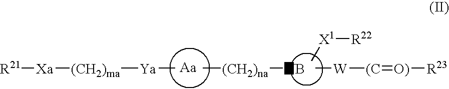
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO2—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

31. The agent according to claim 30, wherein the PPARδ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

32. An ACAT-1mRNA expression-inhibiting agent comprising PPARγ modulator.

33. The agent according to claim 32, wherein the PPARγ modulator is pioglitazone or a salt thereof.

34. The agent according to claim 32, wherein the PPARγ modulator is a compound represented by the formula: 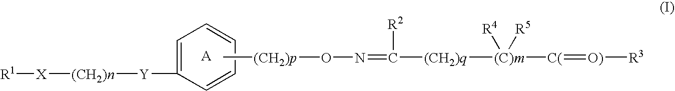
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷ —, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³ is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same of different and each is a hydrogen atom or an optionally substituted hydrocarbon group, or R⁴ and R² may be linked to form a ring, or a salt thereof.

35. The agent according to claim 34, wherein the PPARγ modulator is E-4-[4-(5-methyl-2-phenyl-4-oxazolylmethoxy)benzyloxyimino]-4-phenylbutyric acid or a salt thereof.

36. The agent according to claim 32, wherein the PPARγ modulator is a compound represented by the formula: 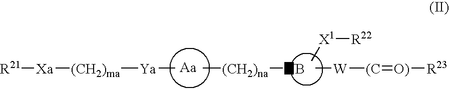
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR 26—, wherein R24 and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO2—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

37. The agent according to claim 36, wherein the PPARγ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.

38. A CEH mRNA expression-promoting agent comprising a PPARγ modulator.

39. The agent according to claim 38, wherein the PPARγ modulator is pioglitazone or a salt thereof.

40. The agent according to claim 38, wherein the PPARγ modulator is a compound represented by the formula: 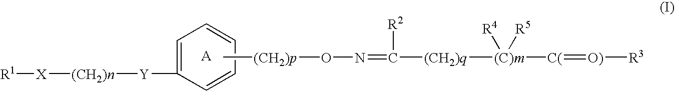
wherein R¹ is a hydrocarbon group or a heterocycle group, each of which is optionally substituted; X is a bond or a group represented by the formula —CO—, —CH(OH)— or —NR⁶—, wherein R⁶ is a hydrogen atom or an optionally substituted alkyl group; n is an integer of 1 to 3; Y is an oxygen atom, a sulfur atom or a group represented by the formula —SO—, —SO₂— or —NR⁷—, wherein R⁷ is a hydrogen atom or an optionally substituted alkyl group; ring A is a benzene ring optionally having further substituent(s); p is an integer of 1 to 8; R² is a hydrogen atom, or a hydrocarbon group or a heterocycle group, each of which is optionally substituted; q is an integer of 0 to 6; m is 0 or 1; R³is a hydroxy group, —OR⁸, wherein R⁸ is an optionally substituted hydrocarbon group, or —NR⁹R¹⁰, wherein R⁹ and R¹⁰ may be the same or different and each is a hydrogen atom, an optionally substituted hydrocarbon group, an optionally substituted heterocycle group or an acyl group, or R⁹ and R¹⁰ may be linked to form a ring; R⁴ and R⁵ may be the same or different and each is a hydrogen atom or an optionally substituted hydrocarbon group, or R⁴ and R² may be linked to form a ring, or a salt thereof.

41. The agent according to claim 40, wherein the PPARγ modulator is E-4-[4-(5-methyl-2-phenyl-4-oxazolylmethoxy)benzyloxyimino]-4-phenylbutyric acid or a salt thereof.

42. The agent according to claim 38, wherein the PPARγ modulator is a compound represented by the formula: 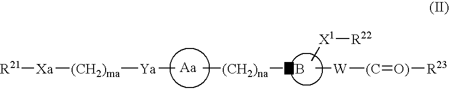
wherein R²¹ is an optionally substituted hydrocarbon group or an optionally substituted heterocycle group;
Xa is a bond, an oxygen atom, a sulfur atom, —CO—, —CS—, —CR²⁴(OR²⁵)— or —NR²⁶—, wherein R²⁴ and R²⁶ are each a hydrogen atom or an optionally substituted hydrocarbon group and R²⁵ is a hydrogen atom or a hydroxy protective group;
ma is an integer of 0 to 3;
Ya is an oxygen atom, a sulfur atom, —SO—, —SO₂—, —NR²⁷—, —CONR²⁷— or —NR²⁷CO—, wherein R²⁷ is a hydrogen atom or an optionally substituted hydrocarbon group;
ring Aa is an aromatic ring optionally having further 1 to 3 substituent(s);
na is an integer of 1 to 8;
ring B is a nitrogen-containing 5-membered heterocycle optionally further substituted with alkyl group(s);
X¹ is a bond, an oxygen atom, a sulfur atom, —SO—, —SO₂—, —O—SO₂— or —NR³⁶—, wherein R³⁶ is a hydrogen atom or an optionally substituted hydrocarbon group;
R²² is a hydrogen atom, an optionally substituted hydrocarbon group or an optionally substituted heterocycle group;
W is a bond or a bivalent hydrocarbon residue having 1 to 20 carbon atom(s);
R²³ is —OR²⁸, wherein R²⁸ is a hydrogen atom or an optionally substituted hydrocarbon group, or —NR²⁹R³⁰, wherein R²⁹ and R³⁰ may be the same or different and each is a hydrogen atom, an optionally substituted hydrocarbon group, an optionally substituted heterocycle group or an optionally substituted acyl group, or R²⁹ and R³⁰ may be linked to form a ring, or a salt thereof.

43. The agent according to claim 42, wherein the PPARγ modulator is
3-[3-ethoxy-1-[4-(2-phenyl-4-thiazolylmethoxy)benzyl]-1H-pyrazol-4-yl]propionic acid;
3-[3-ethoxy-1-[4-(2-phenyl-4-oxazolylmethoxy)benzyl]-1H-pyrazol-4-yl]propionic acid;
3-[3-ethoxy-1-[4-[3-methyl-1-(2-pyridyl)-1H-pyrazol-4-yl]methoxy]benzyl]-1H-pyrazol-4-yl]propionic acid;
3-[1-[4-(5-methyl-2-phenyl-4-oxazolylmethoxy)benzyl]-3-(2-thienyl)-1H-pyrazol-4-yl]propionic acid;
3-[1-[4-(2-phenyl-4-thiazolylmethoxy)benzyl]-3-(2-thienyl)-1H-pyrazol-4-yl]propionic acid; or
3-[1-[4-(2-phenyl-4-oxazolylmethoxy)benzyl]-3-(2-thienyl)-1H-pyrazol-4-yl]propionic acid.


SEQUENCE LISTING (24 sequences)

SEQ ID NO: 1 - DNA, 23 bp, Artificial Sequence
  primer
gctgagctacccaccctatgaac

SEQ ID NO: 2 - DNA, 21 bp, Artificial Sequence
  primer
taatcccctgaacccaaggaa

SEQ ID NO: 3 - DNA, 27 bp, Artificial Sequence
  primer
tgccattttccaaataaagccatgccc

SEQ ID NO: 4 - DNA, 21 bp, Artificial Sequence
  primer
acacctacatgcgtcgcaagt

SEQ ID NO: 5 - DNA, 21 bp, Artificial Sequence
  primer
tcttgccgcttcagtttcttc

SEQ ID NO: 6 - DNA, 28 bp, Artificial Sequence
  primer
tgtgtcctgtcagaagaacagatccgcc

SEQ ID NO: 7 - DNA, 22 bp, Artificial Sequence
  primer
tggaaaatgccaagctgtacct

SEQ ID NO: 8 - DNA, 20 bp, Artificial Sequence
  primer
cggattttgtacctgaggac

SEQ ID NO: 9 - DNA, 26 bp, Artificial Sequence
  primer
ttcatctccctccgcctcattgccta

SEQ ID NO: 10 - DNA, 21 bp, Artificial Sequence
  primer
agcaacagtgtaacaggcgct

SEQ ID NO: 11 - DNA, 21 bp, Artificial Sequence
  primer
acgatggccagctcagtaaag

SEQ ID NO: 12 - DNA, 20 bp, Artificial Sequence
  primer
ctcagaccgcctgcgcgtca

SEQ ID NO: 13 - DNA, 19 bp, Artificial Sequence
  primer
tcaggaggccatgatggtg

SEQ ID NO: 14 - DNA, 19 bp, Artificial Sequence
  primer
cccgtctgccttcatcctt

SEQ ID NO: 15 - DNA, 21 bp, Artificial Sequence
  primer
ccgcgcatctgaagctgcagg

SEQ ID NO: 16 - DNA, 22 bp, Artificial Sequence
  primer
agacctccttcttccaggcttt

SEQ ID NO: 17 - DNA, 23 bp, Artificial Sequence
  primer
gctgcacatcactcagaatttca

SEQ ID NO: 18 - DNA, 26 bp, Artificial Sequence
  primer
tcaccacgaaaatctccagaggcacc

SEQ ID NO: 19 - DNA, 21 bp, Artificial Sequence
  primer
gcagctgctctggttctcatg

SEQ ID NO: 20 - DNA, 23 bp, Artificial Sequence
  primer
ttcttgccagtggtgtaacattg

SEQ ID NO: 21 - DNA, 21 bp, Artificial Sequence
  primer
cggcagatgcagcgaagaggc

SEQ ID NO: 22 - DNA, 18 bp, Artificial Sequence
  primer
agcccttggctgagcaaa

SEQ ID NO: 23 - DNA, 19 bp, Artificial Sequence
  primer
gtcgcaggcagtgaaccat

SEQ ID NO: 24 - DNA, 26 bp, Artificial Sequence
  primer
tgctatcactgctgggtgcaaaacca